(19) United States
(12) Patent Application Publication
(10) Pub. No.: US 2024/0206821 A1
(43) Pub. Date: Jun. 27, 2024

(54) APPARATUS AND A METHOD FOR PREDICTING A PHYSIOLOGICAL INDICATOR

(71) Applicant: nference, Inc., Cambridge, MA (US)

(72) Inventors: Uddeshya Upadhyay, Bengaluru (IN); Sairam Bade, Suryapet (IN); Melwin Babu, Thissur (IN); Ashim Prasad, Mahadevpura (IN); Arjun Puranik, San Jose, CA (US); Rakesh Barve, Bangalore (IN); Murali Aravmudan, Andover, MA (US)

(73) Assignee: nference, Inc., Cambridge, MA (US)

(21) Appl. No.: 18/519,564

(22) Filed: Nov. 27, 2023

(30) Foreign Application Priority Data
Dec. 23, 2022 (IN) 202241074834
Feb. 2, 2023 (IN) 202341006701

(51) Int. Cl. A61B 5/00; A61B 5/318; G16H 50/20

(52) U.S. Cl. CPC A61B 5/7267; A61B 5/318; G16H 50/20

(57) ABSTRACT

An apparatus for predicting a physiological indicator is disclosed. The apparatus comprises a processor and a memory communicatively connected to the at least a processor. The memory instructs the processor to receive a time series input from a user. The memory instructs the processor to predict a physiological indicator as a function of the time series input using a prediction machine learning model. The memory instructs the processor to generate an uncertainty metric as a function of the physiological indicator. Wherein determining the physiological indicator includes receiving a plurality of prediction training data. Determining the physiological indicator includes fitting the plurality of prediction training data with a kernel density estimate. Determining the physiological indicator additionally includes training the prediction machine learning model using a plurality of prediction training data. Training the prediction machine learning model includes, using the kernel density estimate, assigning a weight to each time series input.

### CROSS-REFERENCE TO RELATED APPLICATIONS

This application claims the benefit of priority of Indian Provisional Patent Application Serial No. 202241074834 filed on Dec. 23, 2022, and titled “SYSTEMS AND METHODS FOR RELIABLE UNCERTAINTY-AWARE ACTIVE REGRESSION WITH IMBALANCED MEDICAL TIME SERIES,” which is incorporated by reference herein in its entirety. This application also claims the benefit of priority of Indian Provisional Patent Application Serial No. 202341006701 filed on Feb. 2, 2023, and titled “SYSTEMS AND METHODS FOR RELIABLE UNCERTAINTY-AWARE ACTIVE REGRESSION WITH IMBALANCED MEDICAL TIME SERIES,” which is incorporated by reference herein in its entirety.

### FIELD OF THE INVENTION

The present invention generally relates to the field of analysis of medical time series data. In particular, the present invention is directed to an apparatus and a method for predicting a physiological indicator.

### BACKGROUND

The recognizing the symptoms of rare conditions has long been an issue for medical providers. Identifying these symptoms using computerized diagnostic techniques has presented an additional challenge. Rare conditions traditionally have small sample sizes which have been notoriously difficult for processors to identify additional instances of the rare condition.

### SUMMARY OF THE DISCLOSURE

In an aspect, an apparatus for predicting a physiological indicator is disclosed. The apparatus comprises at least a processor and a memory communicatively connected to the at least a processor. The memory instructs the processor to receive a time series input from a user. The memory instructs the processor to predict a physiological indicator as a function of the time series input using a prediction machine learning model. an uncertainty metric Wherein determining the physiological indicator includes receiving a plurality of prediction training data, wherein the plurality of prediction training data comprises a plurality of time series inputs correlated to examples of physiological indicators as outputs, wherein the examples of physiological indicators comprise continuous value labels. Determining the physiological indicator includes fitting the plurality of prediction training data with a kernel density estimate. Determining the physiological indicator additionally includes training the prediction machine learning model using a plurality of prediction training data, wherein training the prediction machine learning model comprises using the kernel density estimate to weight a contribution of each time series input of the plurality of prediction training data based on an abundance of the corresponding continuous value label.

In another aspect, a method for predicting a physiological indicator is disclosed. The method comprises receiving, using at least a processor, a time series input from a user. The method comprises predicting, using the at least a processor, a physiological indicator as a function of the time series input using a prediction machine learning model. Wherein determining the physiological indicator includes receiving a plurality of prediction training data, wherein the plurality of prediction training data comprises a plurality of time series inputs correlated to examples of physiological indicators as outputs, wherein the examples of physiological indicators comprise continuous value labels. Determining the physiological indicator includes fitting the plurality of prediction training data with a kernel density estimate. Determining the physiological indicator additionally includes training the prediction machine learning model using a plurality of prediction training data, wherein training the prediction machine learning model comprises using the kernel density estimate to weight a contribution of each time series input of the plurality of prediction training data based on an abundance of the corresponding continuous value label. an uncertainty metric

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of an apparatus for predicting a physiological indicator;

FIG. 2 is a block diagram of an exemplary machine-learning process;

FIG. 3 is a block diagram of an exemplary embodiment of a physiological indicator database;

FIG. 4 is a diagram of an exemplary embodiment of a neural network;

FIG. 5 is a diagram of an exemplary embodiment of a node of a neural network;

FIG. 6 is an illustration of an exemplary embodiment of fuzzy set comparison;

FIG. 7 is an illustrative embodiment of a systems for reliable uncertainty-aware active regression with imbalanced medical time series;

FIG. 8 is a flow diagram of an exemplary method for predicting a physiological indicator; and

FIG. 9 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to an apparatus and a method for predicting a physiological indicator is disclosed. The apparatus comprises at least a processor and a memory communicatively connected to the at least a processor. The memory instructs the processor to receive a time series input from a user. The memory instructs the processor to predict a physiological indicator as a function of the time series input using a prediction machine learning model. The memory instructs the processor to generate an uncertainty metric as a function of the physiological indicator. Wherein determining the physiological indicator includes receiving a plurality of prediction training data, wherein the plurality of prediction training data comprises a plurality of time series inputs correlated to examples of physiological indicators as outputs, wherein the examples of physiological indicators comprise continuous value labels. Determining the physiological indicator includes fitting the plurality of prediction training data with a kernel density estimate. Determining the physiological indicator additionally includes training the prediction machine learning model using a plurality of prediction training data, wherein training the prediction machine learning model comprises using the kernel density estimate to weight a contribution of each time series input of the plurality of prediction training data based on an abundance of the corresponding continuous value label. Exemplary embodiments illustrating aspects of the present disclosure are described below in the context of several specific examples.

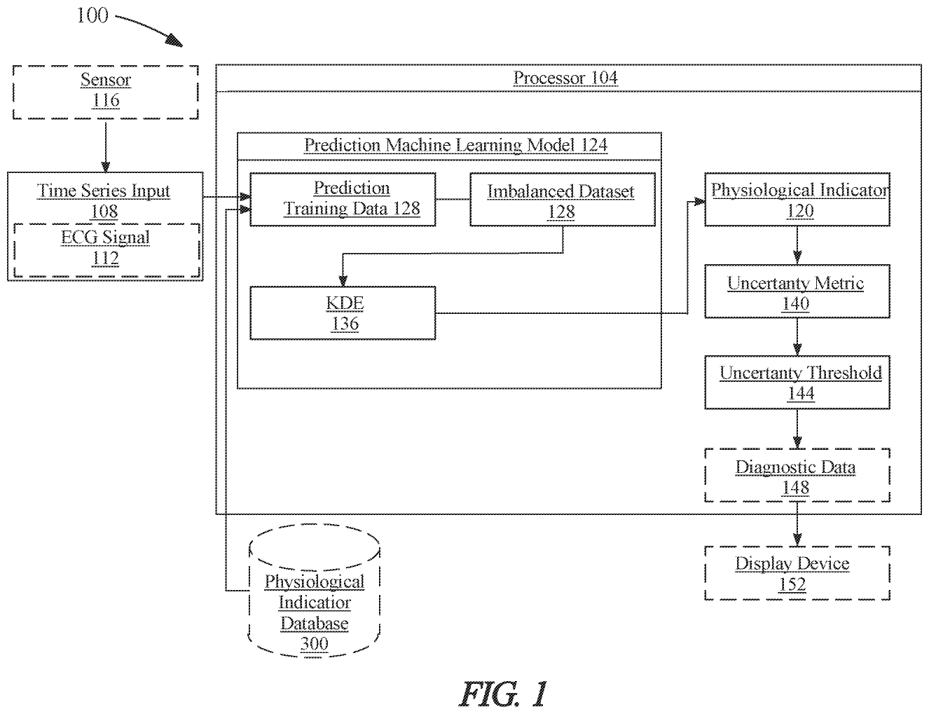

Referring now to FIG. 1, an exemplary embodiment of an apparatus 100 for predicting a physiological indicator is illustrated. Apparatus 100 includes a processor 104. Processor 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Processor 104 may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. Processor 104 may be implemented using a “shared nothing” architecture in which data is cached at the worker, in an embodiment, this may enable scalability of apparatus 100 and/or computing device.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, apparatus 100 includes a memory. Memory is communicatively connected to processor 104. Memory may contain instructions configuring processor 104 to perform tasks disclosed in this disclosure. As used in this disclosure, “communicatively connected” means connected by way of a connection, attachment, or linkage between two or more related which allows for reception and/or transmittance of information therebetween. For example, and without limitation, this connection may be wired or wireless, direct, or indirect, and between two or more components, circuits, devices, systems, apparatus, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example, and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, without limitation, via a bus or other facility for intercommunication between elements of a computing device. Communicative connecting may also include indirect connections via, for example, and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, processor 104 is configured to receive a time series input 108 from a user. As used in the current disclosure, a “time series input” is a type of data that involves the sequential recording of observations or measurements at regular intervals over time. These observations can be related to various physiological, clinical, or health-related parameters, and they are essential for monitoring, diagnosing, and treating medical conditions. Time series inputs 108 are crucial in healthcare for a wide range of applications, including disease monitoring, patient care, and medical research. Time series inputs 108 may include data points that are collected at discrete time intervals, which can be seconds, minutes, hours, days, or even longer, depending on the specific context. This temporal aspect allows healthcare professionals to track changes in a patient's condition over time. Time series inputs 108 may encompass various types of data, such as vital signs (e.g., heart rate, blood pressure, temperature), electrocardiograms (ECG), electroencephalograms (EEG), lab test results, imaging data (e.g., MRI or CT scans), pulse oximetry, blood pressure and more. These diverse data sources provide a comprehensive view of a patient's health. In some cases, time series inputs 108 may not be regularly sampled due to factors like patient compliance or the nature of the measurement. Irregular sampling can present challenges in data analysis and processing. Time series inputs 108 may be used in the diagnostic process to detect anomalies, patterns, or trends that indicate the presence of a medical condition. For example, an abnormal ECG pattern might suggest a cardiac arrhythmia. Time series inputs 108 may include information from a plurality of electronic health records (EHRs). As used in the current disclosure, “electronic health records” are digital records containing a patient's medical history, diagnoses, medications, treatment plans, and other relevant information. EHRs are used by healthcare providers to track and manage patient care. Time series inputs 108 may include information from a plurality of medical imaging data. As used in the current disclosure, “medical imaging data” refers to the visual representations of the internal structures and functions of the human body obtained through various imaging techniques. Medical imaging data may include data associated with X-rays, CT scans, magnetic resonance imaging, ultrasounds, PET scans, nuclear medicine imaging, mammography, fluoroscopy, and the like.

With continued reference to FIG. 1, a time series input 108 may include a plurality of electrocardiogram (ECG) signals 112 from a patient. As used in the current disclosure, a “electrocardiogram signal” is a signal representative of electrical activity of heart. The ECG signal 112 may consist of several distinct waves and intervals, each representing a different phase of the cardiac cycle. These waves may include the P-wave, QRS complex, T wave, U wave, and the like. The P-wave may represent atrial depolarization (contraction) as the electrical impulse spreads through the atria. The QRS complex may represent ventricular depolarization (contraction) as the electrical impulse spreads through the ventricles. The QRS complex may include three waves: Q wave, R wave, and S wave. The T-wave may represent ventricular repolarization (recovery) as the ventricles prepare for the next contraction. The U-wave may sometimes be present after the T wave, it represents repolarization of the Purkinje fibers. The intervals between these waves provide information about the duration and regularity of various phases of the cardiac cycle. The ECG signal can help diagnose various heart conditions, such as arrhythmias, myocardial infarction (heart attack), conduction abnormalities, and electrolyte imbalances. In an embodiment, each sensor 116 may generate an individual ECG signal 112.

With continued reference to FIG. 1, the plurality of electrocardiogram signals may capture a temporal view of cardiac electrical activities. A “temporal view,” as used in the current disclosure, refers to the analysis and visualization of heart-related events and phenomena over time. A temporal view may include patterns, changes, and dynamics of cardiac activity over time. A temporal view may include information surrounding the rhythm of the heart, including the regularity or irregularity of heartbeats. It allows for the identification of various rhythm abnormalities such as tachycardia (fast heart rate), bradycardia (slow heart rate), or arrhythmias (irregular heart rhythms). A temporal view of cardiac activities in three dimensions may refer to a visualization that represents the temporal evolution of cardiac events or phenomena in a three-dimensional space. It provides a comprehensive understanding of how various cardiac activities change over time. The ECG signal 112 may move through the 3D space of the heart over time. The signal does not just move forward in time, it also moves through the physical space of the heart, from SA node through atria, to AV node, and then through the ventricles. Such movement of the electrical signal through the heart's physical space over time can be referred to as “spatiotemporal excitation and propagation” which could be captured by plurality of ECG signals 112. It is a way of observing and analyzing the timing and sequence of the heart's electrical activity as it moves through the physical structure of the heart. In the current case the dimensions may include axis representing time, spatial dimensions, and cardiac activity. By combining the temporal, spatial, and cardiac activity dimensions, the temporal view of cardiac activities in three dimensions allows for a comprehensive visualization and analysis of dynamic changes occurring within the heart. It can be used to study phenomena like electrical conduction, ventricular wall motion, valve function, blood flow dynamics, or the interaction between different regions of the heart. This visualization approach provides valuable insights into the complex temporal dynamics of cardiac activities and aids in understanding cardiac function, pathology, and treatment evaluation.

With continued reference to FIG. 1, the plurality of electrocardiogram signals 112 may be generated using at least a sensor 116. As used in this disclosure, a “sensor” is a device that is configured to detect an input and/or a phenomenon and transmit information related to the detection. Sensor 116 may detect a plurality of data. A plurality of data detected by sensor 11 may include, but is not limited to, electrocardiogram signals 112, heart rate, blood pressure, electrical signals related to the heart, and the like. In one or more embodiments, and without limitation, sensor 116 may include a plurality of sensors 116. In one or more embodiments, and without limitation, sensor 116 may include one or more electrodes, and the like. Electrodes used for an electrocardiogram (ECG) are small sensors or conductive patches that are placed on specific locations on the body to detect and record the electrical signals generated by the heart. Sensor 116 serves as the interface between the body and the ECG machine, allowing for the measurement and recording of the heart's electrical activity. A plurality of sensors 116 may include 10 electrodes used for a standard 12-lead ECG, placed in specific positions on the chest and limbs of the patient. These electrodes are typically made of a conductive material, such as metal or carbon, and are connected to lead wires that transmit the electrical signals to the ECG machine for recording. Proper electrode placement is crucial to ensure accurate signal detection and recording.

With continued reference to FIG. 1, the plurality of sensors 116 may be placed on each limb, wherein there may be at least one sensor 116 on each arm and leg. These sensors 116 may be labeled I, II, III, V1, V2, V3, V4, V5, V6, and the like. For example, Sensor I may be placed on the left arm, Sensor II may be placed on the right arm, and Sensor III may be placed on the left leg. Additionally, a plurality of sensors 116 may be placed on various portions of the patient's torso and chest. For example, a sensor V1 may be placed in the fourth intercostal space at both the right sternal borders and sensor V2 may be fourth intercostal space at both the left sternal borders. A sensor V3 may also be placed between sensors V2 and V4, halfway between their positions. Sensor V4 may be placed in the fifth intercostal space at the midclavicular line. Sensor V5 may be placed horizontally at the same level as sensor V4 but in the anterior axillary line. Sensor V6 may be placed horizontally at the same level as V4 and V5 but in the midaxillary line.

With continued reference to FIG. 1, the plurality of sensors 116 may include augmented unipolar sensors. These sensors 116 may be labeled as aVR, aVL, and aVF. These sensor may be derived from the limb sensors and provide additional information about the heart's electrical activity. These leads are calculated using specific combinations of the limb leads and help assess the electrical vectors in different orientations. For example, aVR may be derived from Sensor II and Sensor III. In another example, aVL may be derived from sensor I and Sensor III. Additionally, aVF may be derived from Lead I and Lead II. The combination of limb sensors, precordial sensors, and augmented unipolar sensors allows for a comprehensive assessment of the heart's electrical activity in three dimensions. These leads capture the electrical signals from different orientations, which are then transformed into transformed coordinates to generate vectorcardiogram (VCG) representing magnitude and direction of electrical vectors during cardiac depolarization and repolarization. Transformed coordinates may include one or more a Cartesian coordinate system (x, y, z), polar coordinate system (r, θ), cylindrical coordinate system (p, q, z), or spherical coordinate system (r, θ, φ). In some cases, transformed coordinates may include an angle, such as with polar coordinates, cylindrical coordinates, and spherical coordinates. In some cases, VCG may be normalized thus permitting full representation with only angles, i.e., angle traversals. In some cases, angle traversals may be advantageously processed with one or more processes, such as those described below and/or spectral analysis.

With continued reference to FIG. 1, processor 104 predicts a physiological indicator 120 as a function of the time series input 108. As used in the current disclosure, a “physiological indicator” is a measurable or observable parameter or characteristic of a biological system, often used to assess the state or function of an organism's physiological processes. These indicators are typically used in medicine, biology, and other life sciences to evaluate health, diagnose diseases, monitor wellness, or understand the functioning of the body. Physiological indicators 120 can include various factors such as vital signs, biochemical markers, anatomical features, or functional parameters. They provide valuable information about an individual's health and well-being. Examples of physiological indicators 120 may include heart rate, respiratory rate, blood pressure, body temperature, blood glucose levels, pulse oximetry, bioimpedance, bioimpedance, hormone levels, the level of potassium in the blood, left ventricular ejection fraction, systolic pressure, and the like. In an embodiment, a physiological indicators 120 may include changes in blood pressure over time, providing information about the cardiovascular system. This typically involves measuring systolic and diastolic pressure. In an additional embodiment, a physiological indicators 120 may include Continuous glucose monitoring generates time series data to track glucose levels, which is critical for managing diabetes.

With continued reference to FIG. 1, a physiological indicator 120 may include a continuous value. As used in the current disclosure, a “continuous value” is a type of numerical data that can take on any real number within a certain range. It is not limited to specific, distinct values and can have an infinite number of possible values within its defined interval. Continuous variables are typically measured, rather than counted, and they can represent a wide range of phenomena, including time, distance, temperature, weight, and many other physical and abstract quantities. In some cases, the examples of physiological indicators may include continuous value labels. As used in the current disclosure, a “continuous value label” refers to a type of label or target variable that takes on a continuous range of values rather than discrete categories. This means that the label can have any real number as its value within a specific range, and it is not limited to distinct classes or categories.

With continued reference to FIG. 1, a physiological indicator 120 may include a measurement of the level of potassium in the blood. There is an indirect relationship between potassium levels in the blood and the time series input 108. Potassium is one of the important electrolytes in the body, and abnormal levels can affect the electrical activity of the heart, which can be detected on an ECG signal 112. Processor 104 identify the if a patent is suffering from hypokalemia or hyperkalemia as a function of changes to the ECG signal 112. Low potassium (hypokalemia) or high potassium (hyperkalemia) levels can lead to changes in the ECG waveform. In an embodiment, low potassium levels can cause the T wave on the ECG signal 112 to become flat or inverted, and it can also lead to U waves. In severe cases, it can cause arrhythmias. In an additional embodiment, high potassium levels can lead to various ECG changes, such as peaked T waves, prolonged PR intervals, widening of the QRS complex, and eventually, it can lead to more serious arrhythmias.

With continued reference to FIG. 1, a physiological indicator 120 may include a measurement of the left ventricular ejection fraction (LVEF.) LVEF is a measure of the heart's pumping efficiency. The time series input 108 for measuring LVEF may include an ECG signal 112 or echocardiograph. An echocardiograph may provide images of the heart's structure and motion, which can be used to calculate LVEF. Processor 104 measure the LVEF using a 12-lead ECG signal 112, which provides information about the heart's electrical activity and rhythm. LVEF may be calculated based on the volume of blood in the left ventricle before and after each heartbeat. ECG signals 112 or other imaging techniques can assess these volumes, enabling the determination of LVEF. In an embodiment, if there is suspicion of reduced LVEF or heart function issues based on the ECG signals 112 or clinical symptoms, additional tests are needed. Processor 104 may provide a notification to the user to indicate that additional testing is needed to identify reduced heart functions.

With continued reference to FIG. 1, processor 104 may generate physiological indicators 120 in real-time. As used in the current disclosure, a “real-time” refers to the immediate or instantaneous processing, analysis, or display of data as it occurs or becomes available, without any noticeable delay involves the continuous collection, processing, and interpretation of data from sensors and instruments. Calculating physiological indicators 120 may involve a continuous collection, processing, and interpretation by apparatus 100 of the time series input 108. To calculate the physiological indicators 120 processor 104 may extract a plurality of features from the time series input 108 in real-time input. Feature extraction is a process that may include identifying and quantifying relevant characteristics in the time series inputs 108. These features can be statistical, spectral, or time-domain measures that capture specific aspects of the physiological signal 120. For example, in ECG signals 112, features might include heart rate, R-R intervals, or wave amplitude. In real-time, the extracted features are continuously analyzed as new data becomes available. This can involve using a trained machine learning model to detect patterns, anomalies, or specific events related to the physiological indicators 120 of interest. The real-time calculation of physiological indicators 120 can trigger alerts or provide feedback to the user or a healthcare provider. For instance, in a medical setting, abnormal values might trigger an alert, or a fitness wearable might provide immediate feedback to the user during exercise. The real-time calculation of physiological indicators 120 may be stored for later analysis and review. This is particularly important in healthcare, where historical data can provide valuable insights into a patient's condition. In some cases, real-time physiological indicators are displayed on a display device for immediate visualization.

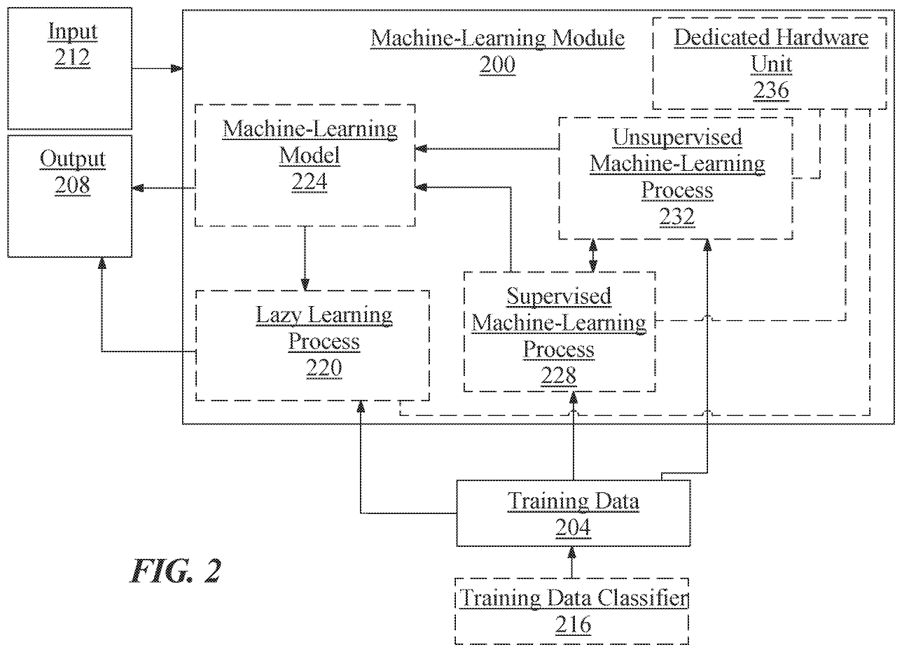

With continued reference to FIG. 1, processor 104 may predict a physiological indicator 120 using a prediction machine-learning model 124. As used in the current disclosure, a “prediction machine-learning model” is a machine-learning model that is configured to generate physiological indicator 120. prediction machine-learning model 124 may be consistent with the machine-learning model described below in FIG. 2. Inputs to the prediction machine-learning model 124 may include time series inputs 108, ECG signals 112, simulated time series inputs, KED 136, examples of imbalanced datasets 136, examples of physiological indicator 120, and the like. Outputs to the prediction machine-learning model 124 may include physiological indicator 120 tailored to the time series input 108. Prediction training data 128 may include a plurality of data entries containing a plurality of inputs that are correlated to a plurality of outputs for training a processor by a machine-learning process. In an embodiment, Prediction training data 128 may include a plurality of time series inputs 108 as inputs correlated to examples of physiological indicators 120 as outputs. In an embodiment, processor 104 may classify data associated with the prediction training data 128 a majority class or a minority class, this may include classifying the time series inputs 108. Prediction training data 128 may be received from database. Prediction training data 128 may contain information about time series inputs 108, ECG signals 112, simulated time series inputs, KED 136, examples of imbalanced datasets 136, examples of physiological indicator 120, and the like. In an embodiment, prediction training data 128 may be iteratively updated as a function of the input and output results of past prediction machine-learning model 124 or any other machine-learning model mentioned throughout this disclosure. The machine-learning model may be performed using, without limitation, linear machine-learning models such as without limitation logistic regression and/or naive Bayes machine-learning models, nearest neighbor machine-learning models such as k-nearest neighbors machine-learning models, support vector machines, least squares support vector machines, fisher's linear discriminant, quadratic machine-learning models, decision trees, boosted trees, random forest machine-learning model, and the like.

With continued reference to FIG. 1, prediction training data 128 may include an imbalanced dataset 132. As used in the current discourse, an “imbalanced dataset” is a dataset where the distribution of class labels is not roughly equal. In other words, it refers to a dataset in which one class (or group) of data is significantly more prevalent than another class or classes. In the current application, the time series inputs 108 may be highly skewed or imbalanced, especially towards normal cases if the prevalence of the abnormal condition or condition of interest is low. For example, a prediction training data 128 may have a high frequency of healthy or normal measurements and a low frequency of unhealthy or abnormal measurements. A heavily skewed or imbalanced dataset 132 can lead to trained prediction machine-learning model 124 that are inaccurate in the prediction or estimate of a continuous value and unable to correctly quantify uncertainty of any such predictions at deployment. This could occur if the dataset used to train the prediction machine-learning model 124 lacks sufficient samples across the range of continuous values. Thus, it is desirable to develop prediction machine-learning model 124 that can accommodate such imbalanced datasets 132 and quantify uncertainty of estimated continuous values (point-estimates). If uncorrected, This imbalance can have a significant impact on the performance of machine learning algorithms, particularly in tasks like classification. In a non-limiting example, consider a binary classification problem where you want to predict whether a user has a rare heart condition based on a time series input 108. If prediction training data 128 includes a dataset with 98% of the users having common heart conditions or no heart conditions and only 2% of user has the rare heart condition, it's an imbalanced dataset 132 because some classes (common heart conditions or no heart conditions) is much more prevalent than the other (rare heart condition). In some cases, machine learning model's that are trained on imbalanced datasets 132 tend to be more accurate when acting on the majority class. They may perform well on the majority class but poorly on the minority class. The algorithms might not have enough examples of the minority class to learn its characteristics effectively, leading to poor generalization. Additionally, in imbalanced datasets 132 accuracy of the machine learning may not a suitable metric for evaluating the performance of models on imbalanced datasets 132 because a simple model that predicts the majority class for all instances can achieve high accuracy.

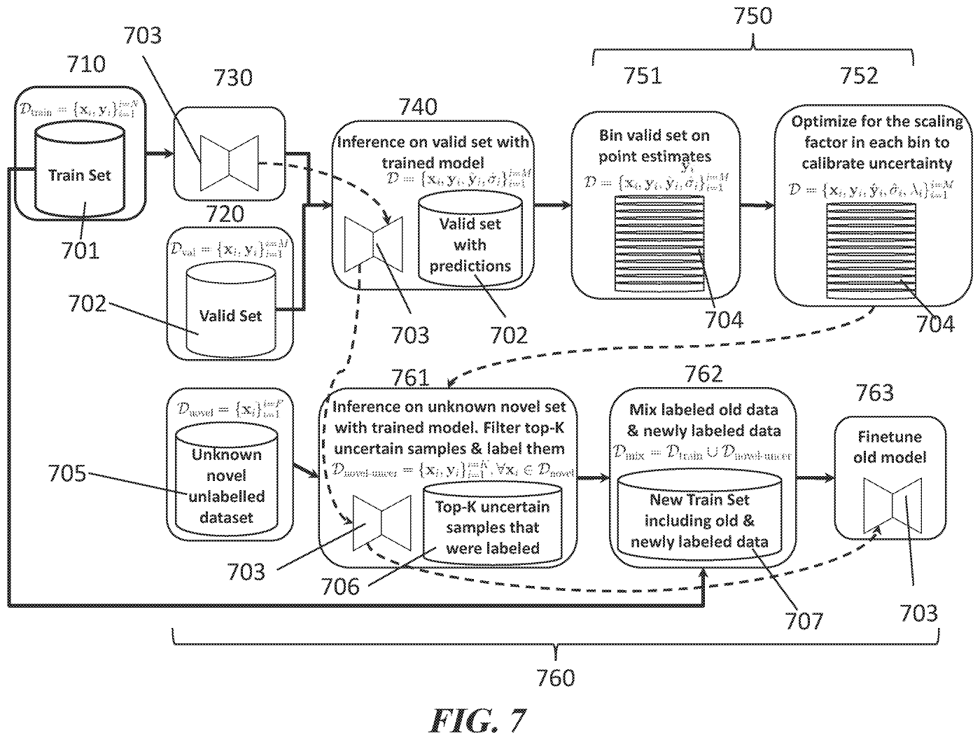

With continued reference to FIG. 1, processor 104 may compensate for the imbalanced dataset 132 using one or more methods. Compensating for an imbalanced dataset 132 may include identifying a way to appropriately represent the minority class within the imbalanced dataset 132 when training a machine-learning model. In a first embodiment, processor 104 may compensate for the imbalanced dataset 132 by modifying prediction training data 128. In a non-limiting example, processor 104 may adjust the weights assigned to both members of the minority class and the majority class to compensate for the imbalanced dataset 132. In an embodiment, processor 104 may address class imbalance is by using a weighted loss function. In this approach, you assign different weights to each class, typically giving more weight to the minority class when training a machine-learning model. This means that errors in the minority class have a more significant impact on the loss, which encourages the model to pay more attention to it. The loss function can be defined as something like weighted cross-entropy or weighted hinge loss. Adjusting the weights within the imbalanced dataset 132 may be discussed in greater detail herein below in FIG. 7.

With continued reference to FIG. 1, processor 104 may compensate for the imbalanced dataset 132 using various methods. In an embodiment, processor 104 may assign class-specific weights to each class to improve the training of a machine-learning model using the imbalanced dataset 132. By assigning higher weights to the minority class, processor 104 may address the discrepancies from the volume of the minority class to the majority class. In another embodiment, processor may compensate for the imbalanced dataset 132 by adjusting the decision making threshold of the prediction machine-learning model 124. In an non-limiting example, prediction machine-learning model 124 may use a threshold of 0.5 to predict physiological indicators 120. Adjusting this threshold can make the model more sensitive to the minority class. This is especially useful when using probability-based models. In a third embodiment, processor 104 may modify the prediction machine-learning model 124 to assign different misclassification costs to different classes. By assigning a higher cost to misclassifying the minority class, you can make the model more sensitive to it.

With continued reference to FIG. 1, processor 104 may fit the plurality of prediction training data with a kernel density estimate (KDE) 136. As used in the current disclosure, a “kernel density estimate” is a non-parametric statistical method used to estimate the probability density function (PDF) of a continuous random variable. It is a way to visualize and approximate the underlying distribution of a dataset. KDE 136 is particularly useful when you have data points and want to understand the shape of the data's distribution without assuming any specific parametric form, such as a normal distribution. In KDE 136, a “kernel” is a smooth and continuous function that is typically symmetric and centered at a data point. When generating KDE 136, for each data point within the prediction training data 128 processor 104 may place a kernel function centered at that point. Processor 104 may then take a sum of the kernel functions at each point in the domain of interest to estimate the PDF. The KDE 136 at a particular point x is computed as the sum of the kernel values evaluated at x for all data points. In some cases, the KDE 136 is normalized so that the integral (or sum) over the entire domain equals 1, ensuring that it represents a valid probability density function. The KDE 136 estimate provides a smoothed representation of the data's distribution, allowing you to see its shape and key features, such as peaks, modes, and tails. It's a powerful tool for data visualization and can be used for various purposes, including density estimation, data analysis, and hypothesis testing.

With continued reference to FIG. 1, processor 104 may be configured to manipulate the selection of bandwidth and kernel functions according to the specific application. Processor 104 may select at least one kernel function based on the Prediction training data 128. Examples of kernel functions may include the Gaussian (normal) kernel, Epanechnikov kernel, and others. The choice of kernel function affects the shape and smoothness of the estimated PDF. When selecting the bandwidth (h) for the KDE 136 processor 104 may consider the smoothness and the potential noise within the dataset. Bandwidth is a crucial parameter in KDE 136. It controls the width of the kernel and, in turn, the smoothness of the estimate. A smaller bandwidth results in a more detailed but noisy estimate, while a larger bandwidth results in a smoother but less detailed estimate. Bandwidth selection is important, and various methods can be used to choose an appropriate bandwidth, such as cross-validation.

With continued reference to FIG. 1, the KDE 136 may be used to create separate plots for the majority and minority classes to help a user to visualize the probability density for each class. The KDE 136 may also help the user identify KDE which features are more discriminative for separating the classes. Features with distinct density peaks or regions in the KDE plot for one class compared to the other may be important for classification. In some cases, KDE 136 may be used to select the optimal probability thresholds for classification. For instance, you can choose a threshold that maximizes the F1-score or another relevant performance metric based on the KDE estimated densities.

With continued reference to FIG. 1, machine learning plays a crucial role in enhancing the function of software for generating an prediction machine-learning model 124. This may include identifying patterns within the time series input 108 that lead to changes in the capabilities and type of the prediction machine-learning model 124. By analyzing vast amounts of data related to biological data, machine learning algorithms can identify patterns, correlations, and dependencies that contribute to generating the prediction machine-learning model 124. These algorithms can extract valuable insights from various sources, including text, document, audio, and other multimodal data associated with the time series input 108. By applying machine learning techniques, the software can generate the prediction machine-learning model 124 extremely accurately. Machine learning models may enable the software to learn from past collaborative experiences of the entities and iteratively improve its training data over time.

With continued reference to FIG. 1, processor 104 may be configured to update the training data of the prediction machine-learning model 124 using user inputs. Prediction machine-learning model 124 may use user input to update its training data, thereby improving its performance and accuracy. In embodiments, the prediction machine-learning model 124 may be iteratively updated using input and output results of the prediction machine-learning model 124. The prediction machine-learning model 124 may then be iteratively retrained using the updated machine-learning model. For instance, and without limitation, prediction machine-learning model 124 may be trained using a first training data from, for example, and without limitation, training data from a user input or database. The prediction machine-learning model 124 may then be updated by using previous inputs and outputs from the prediction machine-learning model 124 as second training data to then train a second machine learning model or a second iteration of the prediction machine-learning model 124 This process of updating the prediction machine-learning model 124 may be continuously done to create subsequent a second set of inquiries 120 which may be used to prompt the user to provide a second set of inquiry responses 156. The second set on inquiry responses may be used as training data for specifically training the inquiry machine-learning model 124. The additional training data provided by second set of inquiries 120 may be used to improve the speed and accuracy of the prediction machine-learning model 124. When users interact with the software, their actions, preferences, and feedback provide valuable information that can be used to refine and enhance the model. This user input is collected and incorporated into the training data, allowing the machine learning model to learn from real-world interactions and adapt its predictions accordingly. By continually incorporating user input, the model becomes more responsive to user needs and preferences, capturing evolving trends and patterns. This iterative process of updating the training data with user input enables the machine learning model to deliver more personalized and relevant results, ultimately enhancing the overall user experience. The discussion within this paragraph may apply to both the prediction machine-learning model 124 and/or any other machine-learning model/classifier discussed herein.

With continued reference to FIG. 1, retraining the prediction machine-learning model may further include labeling the subset of novel time-series inputs. For the purposes of this disclosure, “novel time-series inputs” refers to time-series data that has not yet been used for training the prediction machine-learning model. Labeling the novel time series inputs may refer to the process of assigning meaningful and relevant categories or values to data points. In the case of time-series inputs, labeling typically involves associating each data point in the time series with a specific class, category, or value (i.e. a physiological value). This labeling step is essential for supervised learning, where a model learns from labeled examples to make predictions. In some cases, the prediction machine-learning model may be retrained using the labeled subset of novel time-series data. In an embodiment, labeling a subset of novel time-series data may include labeling the subset of novel time-series data using active learning. Active learning is a machine learning technique that involves an iterative process of selecting the most informative or uncertain data points for labeling. Instead of randomly labeling data points, active learning algorithms intelligently choose the data points that are expected to provide the most valuable information for improving the model's performance. These algorithms may use uncertainty metrics, model confidence, or other strategies to decide which data points to label next. In some cases, active learning may be a semi-supervised process where the model starts with a small labeled dataset. Instead of passively learning from the entire labeled dataset, active learning algorithms select which unlabeled data points to query for labels. The goal is to choose the most informative or uncertain data points for labeling. Active learning may aim to reduce the amount of labeled data required. It focuses on selecting the most informative data points for labeling, potentially leading to a more efficient labeling process.

Incorporating the user feedback may include updating the training data by removing or adding correlations of user data to a path or resources as indicated by the feedback. Any machine-learning model as described herein may have the training data updated based on such feedback or data gathered using a web crawler as described above. For example, correlations in training data may be based on outdated information wherein, a web crawler may update such correlations based on more recent resources and information.

With continued reference to FIG. 1, processor 104 may use user feedback to train the machine-learning models and/or classifiers described above. For example, machine-learning models and/or classifiers may be trained using past inputs and outputs of classifier. In some embodiments, if user feedback indicates that an output of classifier was “bad,” then that output and the corresponding input may be removed from training data used to train classifier, and/or may be replaced with a value entered by, e.g., another value that represents an ideal output given the input the machine learning model originally received, permitting use in retraining, and adding to training data; in either case, classifier may be retrained with modified training data as described in further detail below. In some embodiments, training data of classifier may include user feedback.

With continued reference to FIG. 1, in some embodiments, an accuracy score may be calculated for classifier using user feedback. For the purposes of this disclosure, “accuracy score,” is a numerical value concerning the accuracy of a machine-learning model. For example, the accuracy/quality of the outputted prediction machine-learning model 124 may be averaged to determine an accuracy score. In some embodiments, an accuracy score may be determined for how well the prediction machine learning model 124 is able to identify the minority class. Accuracy score or another score as described above may indicate a degree of retraining needed for a machine-learning model such as a classifier; processor 104 may perform a larger number of retraining cycles for a higher number (or lower number, depending on a numerical interpretation used), and/or may collect more training data for such retraining. The discussion within this paragraph and the paragraphs preceding this paragraph may apply to both the prediction machine-learning model 124 and/or any other machine-learning model/classifier mentioned herein.

With continued reference to FIG. 1, generate an uncertainty metric 140 as a function of the physiological indicator 120. As used in the current disclosure, an “uncertainty metric” is a quantitative measure used to assess and communicate the degree of uncertainty associated with a particular data point. Uncertainty refers to the lack of precision or confidence in a specific data point, measurement, prediction, or model output, such as a physiological indicator 120. An uncertainty metric 140 quantifies the degree to which the information being assessed is subject to variability, error, or doubt. Higher uncertainty might indicate a unfavorable uncertainty metric 140, whereas a lower uncertainty may indicate a favorable uncertainty metric 140. An uncertainty metric 140 may provide a numerical or qualitative measure of this uncertainty, allowing processor 104 to gauge the reliability and trustworthiness of the physiological indicator 120. Processor 104 may normalize the uncertainty metrics 140 to ensure they fall within a consistent range (e.g., 0-1, 0-10, 0-100, and the like) for ease of interpretation. Additionally, an uncertainty metric 140 may be reflected as a linguistic variable. A linguistic variable may include terms such as “Highly Certain,” “Moderately Certain,” “Moderately Uncertain,” “Highly Uncertain,” “No Response Available,” and the like. A linguistic variable may be associated with a numerical score associated with the uncertainty metric 140. In a non-limiting embodiment, the linguistic variable of “Highly Certain” may be associated with a numerical score above 97%. Processor 104 may adjust uncertainty metric 140 based on known clinical factors. For instance, if a time series input 108 aligns strongly with a particular physiological indicator 120, it could result in a favorable uncertainty metric 140. In an embodiment, an uncertainty metrics 140 may involve defining a range or interval within which the true value is likely to fall. For example, if it is known that a heart condition only appears in only one patient in a million, processor 104 may have a lower confidence in the diagnosis based on the statistical probability. In an embodiment, processor 104 may employ fuzzy logic to represent and quantify uncertainty by allowing values to belong to a range of possible memberships, indicating the degree to which an element belongs to a particular category. An uncertainty metric 140 may be generated by an uncertainty machine learning model as a described herein below. Processor 104 may generate an uncertainty metric 140 based on the model's prediction confidence, such as variance, entropy, or Bayesian probabilities.

With continued reference to FIG. 1, processor 104 may be configured to calibrate an uncertainty metric for the prediction machine-learning model. Calibration may refer to the process of fine-tuning or adjusting the model's predictions to align more closely with the actual probabilities. A well-calibrated model is one where, for instance, if it predicts a 70% probability for a certain event, that event actually occurs about 70% of the time. Calibrating the uncertainty metric of the prediction machine-learning model may include receiving a set of validation data. As used in the current disclosure, a “a set of validation data” is a separate portion of the data that the model hasn't seen during training. It is used to assess the model's performance and, in this case, to calibrate the uncertainty metric. Processor 104 may sort each datapoint of the validation set into a plurality of hyperfine bins as a function of a continuous value. As used in the current disclosure, “hyperfine bins” is a grouping of the data points into a set of very fine or detailed bins. These bins are may be organized based on the values of the continuous variable. Each bin corresponds to a specific range or interval of continuous values. Processor 104 may additionally determine a bin-wise scaling factor for each of the plurality of hyperfine bins. As used in the current disclosure, “bin-wise scaling factors” refers to a factor or multiplier associated with each individual hyperfine bin. It may be used to adjust or scale the data within each bin. The scaling factor can be unique to each bin and is typically determined based on some specific criteria or algorithm.

With continued reference to FIG. 1, calibration of the uncertainty metric may include temperature scaling. As used in the current disclosure, “temperature scaling” is a technique used in the calibration of probability outputs from a machine learning model, particularly in the context of classification tasks. The goal of temperature scaling is to adjust the confidence scores or probabilities generated by a model to make them better reflect the true uncertainty or reliability of the model's predictions. This technique is often used to improve the calibration of deep neural networks, especially in cases where model confidence scores do not align well with actual probabilities. Temperature scaling introduces a hyperparameter known as the “temperature” (T). The temperature is a positive scalar value that is applied to the logits (raw scores) before they are passed through the softmax function. By adjusting the temperature, processor 104 can control the sharpness or spread of the probability distribution. A higher temperature makes the distribution more uniform, while a lower temperature makes the distribution sharper. In an embodiment, high temperature may smooth the distribution, reducing confidence in predictions. It makes the model less certain about its output probabilities. In an additional embodiment, low temperature may sharpen the distribution, increasing confidence in predictions. It makes the model more certain about its output probabilities. The temperature parameter may be adjusted based on a validation dataset. The goal is to find the temperature that minimizes the difference between the predicted probabilities and the true probabilities observed in the calibration dataset.

With continued reference to FIG. 1, processor 104 may choose a subset of the novel time-series inputs, wherein choosing the subset comprises choosing values of the set of novel-time series inputs as a function of the uncertainty metric. In an embodiment, processor may user uncertainty metrics 140 to determine what values the model exhibits the most uncertainty for. Then selecting those values to further train the model. Instead of using all available data, processor 104 may be configured to focus on a specific portion of the imbalanced dataset. The process of choosing this subset is guided by the portion of the imbalanced dataset which the processor 104 has the most uncertainty in. Selecting a subset of the novel time series input may be discussed in greater detail herein below in FIG. 7.

With continued reference to FIG. 1, processor 104 may compare the uncertainty metric 140 to an uncertainty threshold 144. As used in the current disclosure, a “uncertainty threshold” is a predefined level of confidence or probability that is used to make decisions or take actions based on the output of a machine learning model. An uncertainty metric 140 may serve as a cutoff point or boundary beyond which the model's predictions or classifications are considered reliable and actionable. In an embodiment, when the uncertainty metric 140 surpasses the defined threshold, the model's output is accepted as accurate and trustworthy. Conversely, if the uncertainty metric 140 falls below this threshold, the prediction may be deemed less reliable, and further review, manual intervention, or an alternative approach may be warranted. In an embodiment, processor 104 may retrain the prediction machine-learning model 124 if the uncertainty metric 140 fails to reach the uncertainty threshold 144. Retraining may include re-compensating for the imbalanced dataset 132. This may include adding and/or modifying the synthetic time series inputs. The past input, outputs, compensation attempts may be used as training data for the prediction machine learning model. This may be done with a goal of refining the inputs into the prediction machine-learning model 124 to further refine the physiological indicator 120. In a non-limiting example, a uncertainty threshold 144 could be set at 0.93. If the prediction machine-learning model 124 predicts a physiological indicator 120 with an uncertainty metric 140 of 0.8, it will not exceed the threshold, indicating low confidence in the result.

With continued reference to FIG. 1, processor 104 may generate uncertainty metric 140 using an uncertainty machine-learning model. As used in the current disclosure, an “uncertainty machine-learning model” is a machine-learning model that is configured to generate uncertainty metric 140. uncertainty machine-learning model may be consistent with the machine-learning model described below in FIG. 2. Inputs to the uncertainty machine-learning model may include time series input 108, ECG signals 112, synthetic time series inputs, imbalanced dataset 132, physiological indicator 120, KDE 136, prediction training data 128, examples of uncertainty metric 140, and the like. Outputs to the uncertainty machine-learning model may include uncertainty metric 140 tailored to the physiological indicator 120. Additionally uncertainty machine learning model may be configured to compare the uncertainty metric 140 to the uncertainty threshold 144. Uncertainty training data may include a plurality of data entries containing a plurality of inputs that are correlated to a plurality of outputs for training a processor by a machine-learning process. In an embodiment, uncertainty training data may include a plurality of physiological indicators 120 correlated to examples of uncertainty metrics 140. Uncertainty training data may be received from a database. Uncertainty training data may contain information about time series input 108, ECG signals 112, synthetic time series inputs, imbalanced dataset 132, physiological indicator 120, KDE 136, prediction training data 128, examples of uncertainty metric 140, and the like. In an embodiment, uncertainty training data may be iteratively updated as a function of the input and output results of past uncertainty machine-learning model or any other machine-learning model mentioned throughout this disclosure. The machine-learning model may be performed using, without limitation, linear machine-learning models such as without limitation logistic regression and/or naive Bayes machine-learning models, nearest neighbor machine-learning models such as k-nearest neighbors machine-learning models, support vector machines, least squares support vector machines, fisher's linear discriminant, quadratic machine-learning models, decision trees, boosted trees, random forest machine-learning model, and the like.

With continued reference to FIG. 1, processor 104 may accept the physiological indicator 120 as a function of the comparison of the uncertainty metric 140 to the uncertainty threshold 144. The uncertainty metric is then compared to the uncertainty threshold. The comparison may involve assessing whether the calculated uncertainty falls within the acceptable range defined by the threshold. This comparison is typically done quantitatively, but it can also include a qualitative evaluation if applicable. If the uncertainty metric 140 falls within or is greater than the established threshold, the physiological indicator 120 is considered reliable, and it can be accepted as a valid indicator. This means that the level of uncertainty is deemed acceptable for the intended purpose. Alternatively, if the calculated uncertainty metric 140 fails to meet the defined uncertainty threshold 144, caution is warranted. In this case, the physiological indicator 120 is considered unreliable or less trustworthy. It may be necessary to take corrective actions, repeat the measurement, or seek further verification to reduce the uncertainty. In an embodiment, a feedback loop may be triggered by failing to meet the uncertainty threshold 144. This may serve as a critical mechanism for improving the reliability and accuracy of the output of the prediction machine-learning model 124. When an uncertainty metric 140 fails to meet the predefined threshold of acceptable uncertainty, it signals the need for corrective action. This feedback initiates a continuous improvement cycle where efforts are made to identify and rectify the sources of uncertainty, enhance measurement techniques, or recalibrate instruments to bring the measurement within the acceptable range. As a result, this iterative process ensures that data quality is maintained, mitigating risks associated with unreliable information and contributing to the overall robustness of decision-making and quality control protocols in fields such as healthcare, scientific research, and engineering.

With continued reference to FIG. 1, the memory may instruct the processor to a generate diagnostic data 148 as a function of the acceptance of the physiological indicator 120. As used in the current disclosure, “diagnostic data” is data associated with the diagnosis of a user's medical condition or health issue. Processor 104 may generate diagnostic data as a function of the collected and analyzed data from the time series input 108 and physiological indicator 120. Diagnostic data 148 may identify the condition of or name of the disease the user is suffering from based on known conditions/symptoms of the user. In a non-limiting example, a abnormalities within an ECG signal 116 may indicate the presence of abnormal rhythms (arrhythmias), such as atrial fibrillation or ventricular tachycardia. To generate the diagnostic data 148 processor 104 may preprocess the time series input 108 and physiological indicator 120 The raw time series input 108 and physiological indicator 120 may contain noise, baseline wander, and artifacts. Pre-processing steps involve filtering the signal to remove noise, such as powerline interference and muscle artifacts. It also includes baseline drift correction. If multiple physiological parameters are measured simultaneously, ensuring that the data is properly synchronized in time is crucial for accurate analysis. The collected and preprocessed data may be typically stored in a secure database or storage system. This allows for easy retrieval and long-term archiving. Relevant features may be extracted from the physiological indicator 120, such as peaks, trends, or statistical metrics. These features can help in characterizing the physiological parameter. In an embodiment, algorithms and machine learning techniques can be applied to recognize patterns and anomalies within the data. For instance, identifying irregular heart rhythms or unusual temperature fluctuations. The processor 104 may compare the collected physiological indicator 120 to reference data or established norms to assess whether the measurements fall within expected ranges. Processors 104 may use specialized diagnostic algorithms, which consider multiple parameters and historical data to make informed assessments. These algorithms can be based on clinical guidelines or specific domain knowledge. In some embodiments, if an abnormality or diagnostic insight is detected, the processor may generate a diagnostic report. This report typically may include information about the patient's condition, potential diagnoses, and recommended actions. If a critical condition or an emergency is detected, the processor can trigger alerts and notifications to healthcare providers, patients, or relevant stakeholders. In some embodiments, integrate the diagnostic data 148 and/or the physiological indicator 120 into a healthcare system, such as Electronic Health Records (EHR) systems, to share diagnostic information and ensure continuity of care.

Still referring to FIG. 1, processor 104 may be configured to display the accepted physiological indicator 120 using a display device 156. As used in the current disclosure, a “display device” is a device that is used to display a plurality of data and other digital content. A display device 156 may include a user interface. A “user interface,” as used herein, is a means by which a user and a computer system interact; for example through the use of input devices and software. A user interface may include a graphical user interface (GUI), command line interface (CLI), menu-driven user interface, touch user interface, voice user interface (VUI), form-based user interface, any combination thereof, and the like. A user interface may include a smartphone, smart tablet, desktop, or laptop operated by the user. In an embodiment, the user interface may include a graphical user interface. A “graphical user interface (GUI),” as used herein, is a graphical form of user interface that allows users to interact with electronic devices. In some embodiments, GUI may include icons, menus, other visual indicators, or representations (graphics), audio indicators such as primary notation, and display information and related user controls. A menu may contain a list of choices and may allow users to select one from them. A menu bar may be displayed horizontally across the screen such as pull-down menu. When any option is clicked in this menu, then the pulldown menu may appear. A menu may include a context menu that appears only when the user performs a specific action. An example of this is pressing the right mouse button. When this is done, a menu may appear under the cursor. Files, programs, web pages and the like may be represented using a small picture in a graphical user interface. For example, links to decentralized platforms as described in this disclosure may be incorporated using icons. Using an icon may be a fast way to open documents, run programs etc. because clicking on them yields instant access. Information contained in user interface may be directly influenced using graphical control elements such as widgets. A “widget,” as used herein, is a user control element that allows a user to control and change the appearance of elements in the user interface. In this context a widget may refer to a generic GUI element such as a check box, button, or scroll bar to an instance of that element, or to a customized collection of such elements used for a specific function or application (such as a dialog box for users to customize their computer screen appearances). User interface controls may include software components that a user interacts with through direct manipulation to read or edit information displayed through user interface. Widgets may be used to display lists of related items, navigate the system using links, tabs, and manipulate data using check boxes, radio boxes, and the like.

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm instantiated in hardware or software logic, data structures, and/or functions that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative example time series inputs 108 as inputs correlated to physiological indicators 120 as outputs.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 200 may generate a classifier using a classification algorithm, defined as a processes whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to time series inputs 108 to a majority class or a minority class.

With further reference to FIG. 2, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Still referring to FIG. 2, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may identified as having poor quality data, where “poor quality” is defined as having a signal to noise ratio below a threshold value.

As a non-limiting example, and with further reference to FIG. 2, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 2, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 136. Processor may interpolate the low pixel count image to convert the 100 pixels into 136 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units

In some embodiments, and with continued reference to FIG. 2, computing device, processor, and/or module may down-sample elements of a training example to a desired lower number of data elements. As a non-limiting example, a high pixel count image may have 256 pixels, however a desired number of pixels may be 136. Processor may down-sample the high pixel count image to convert the 256 pixels into 136 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include time series inputs 108 inputs as described above as inputs, physiological indicators 120 as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

With further reference to FIG. 2, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 2, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 232 may not require a response variable; unsupervised processes 232 may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 2, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 2, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 2, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 2, one or more processes or algorithms described above may be performed by at least a dedicated hardware unit 236. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 236 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 236 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 236 to perform one or more operations described herein, such as evaluation of model and/or algorithm outputs, one-time or iterative updates to parameters, coefficients, weights, and/or biases, and/or any other operations such as vector and/or matrix operations as described in this disclosure.

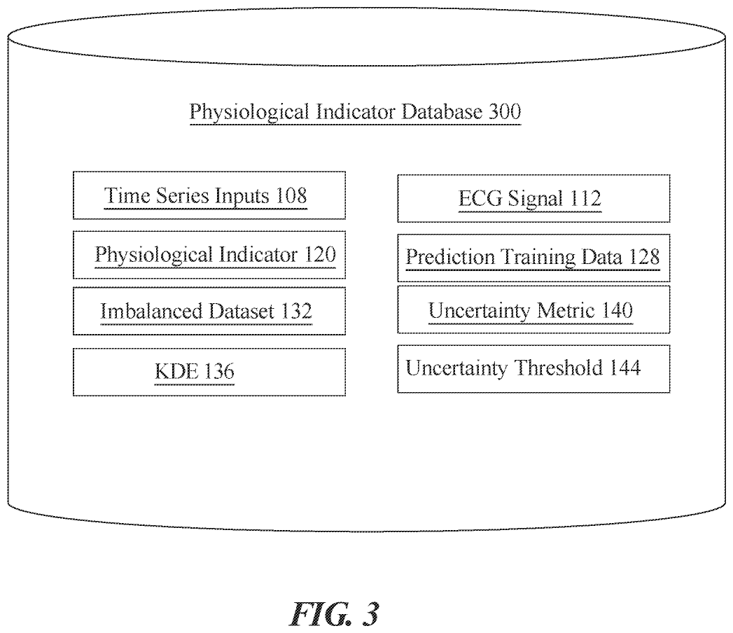

Now referring to FIG. 3, an exemplary physiological indicator database 300 is illustrated by way of block diagram. In an embodiment, any past or present versions of any data disclosed herein may be stored within the physiological indicator database 300 including but not limited to: time series inputs 108, ECG signals 112, simulated time series inputs, KED 136, imbalanced datasets 136, physiological indicator 120, prediction training data 128, uncertainty metrics, and the like. Processor 104 may be communicatively connected with physiological indicator database 300. For example, in some cases, database 300 may be local to processor 104. Alternatively or additionally, in some cases, database 300 may be remote to processor 104 and communicative with processor 104 by way of one or more networks. Network may include, but not limited to, a cloud network, a mesh network, or the like. By way of example, a “cloud-based” system, as that term is used herein, can refer to a system which includes software and/or data which is stored, managed, and/or processed on a network of remote servers hosted in the “cloud,” e.g., via the Internet, rather than on local severs or personal computers. A “mesh network” as used in this disclosure is a local network topology in which the infrastructure processor 104 connects directly, dynamically, and non-hierarchically to as many other computing devices as possible. A “network topology” as used in this disclosure is an arrangement of elements of a communication network. Physiological indicator database 300 may be implemented, without limitation, as a relational database, a key-value retrieval database such as a NOSQL database, or any other format or structure for use as a database that a person skilled in the art would recognize as suitable upon review of the entirety of this disclosure. Physiological indicator database 300 may alternatively or additionally be implemented using a distributed data storage protocol and/or data structure, such as a distributed hash table or the like. Physiological indicator database 300 may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

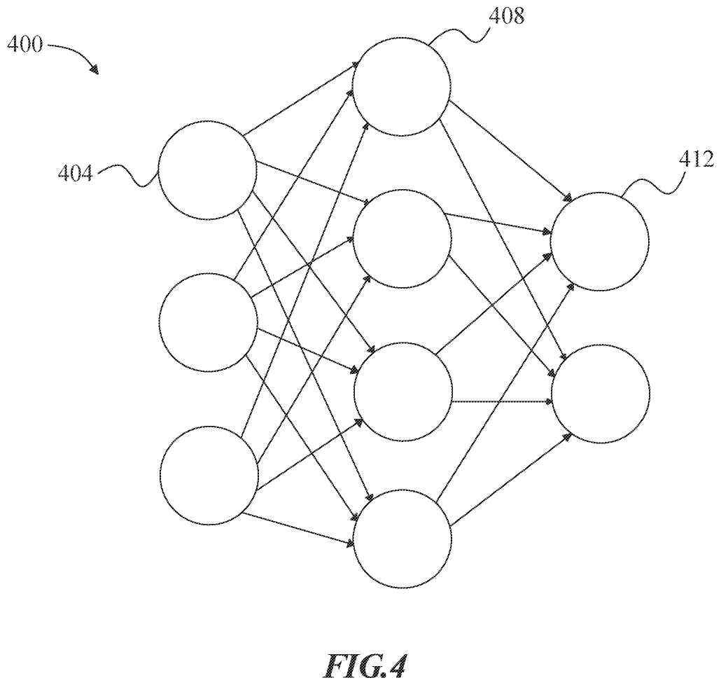

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

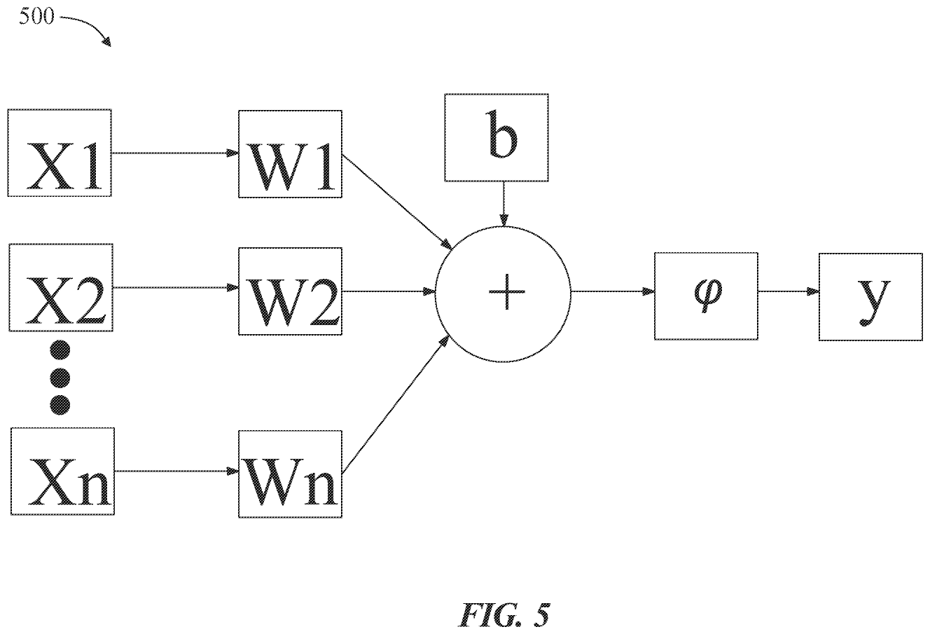

Referring now to FIG. 5, an exemplary embodiment of a node of a neural network is illustrated. A node may include, without limitation, a plurality of inputs xᵢ that may receive numerical values from inputs to a neural network containing the node and/or from other nodes. Node may perform a weighted sum of inputs using weights wᵢ, that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ, applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ, may be determined by training a neural network using training data, which may be performed using any suitable process as described above.

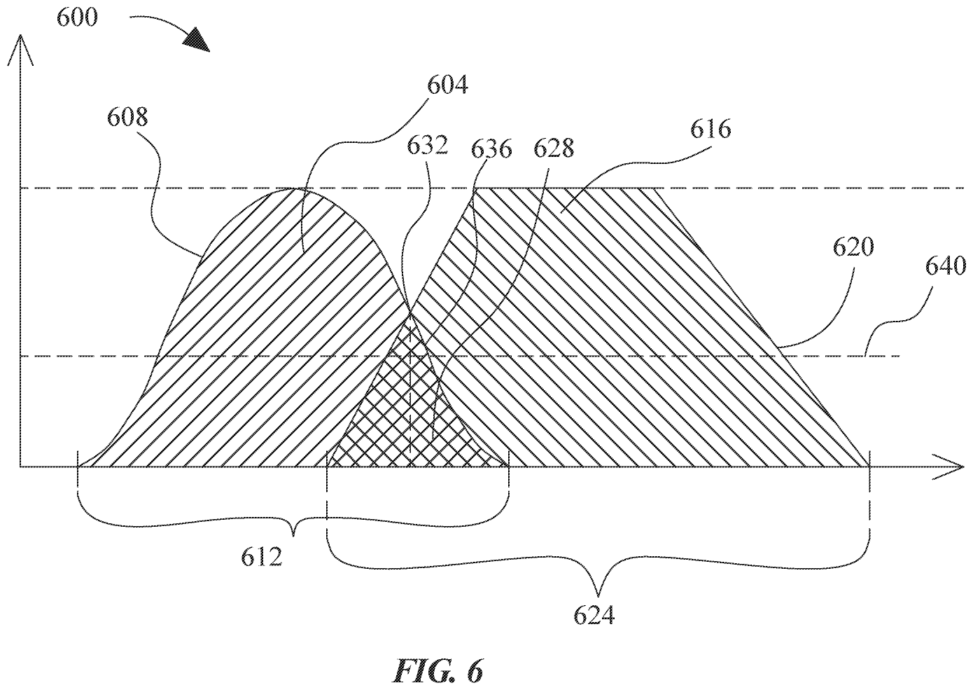

Now referring to FIG. 6, an exemplary embodiment of fuzzy set comparison 600 is illustrated. In a non-limiting embodiment, the fuzzy set comparison. In a non-limiting embodiment, fuzzy set comparison 600 may be consistent with fuzzy set comparison in FIG. 1. In another non-limiting the fuzzy set comparison 600 may be consistent with the name/version matching as described herein. For example and without limitation, the parameters, weights, and/or coefficients of the membership functions may be tuned using any machine-learning methods for the name/version matching as described herein. In another non-limiting embodiment, the fuzzy set may represent time series inputs 108 and physiological indicator 120 from FIG. 1.

Alternatively or additionally, and still referring to FIG. 6, fuzzy set comparison 600 may be generated as a function of determining the data compatibility threshold. The compatibility threshold may be determined by a computing device. In some embodiments, a computing device may use a logic comparison program, such as, but not limited to, a fuzzy logic model to determine the compatibility threshold and/or version authenticator. Each such compatibility threshold may be represented as a value for a posting variable representing the compatibility threshold, or in other words a fuzzy set as described above that corresponds to a degree of compatibility and/or allowability as calculated using any statistical, machine-learning, or other method that may occur to a person skilled in the art upon reviewing the entirety of this disclosure. In some embodiments, determining the compatibility threshold and/or version authenticator may include using a linear regression model. A linear regression model may include a machine learning model. A linear regression model may map statistics such as, but not limited to, frequency of the same range of version numbers, and the like, to the compatibility threshold and/or version authenticator. In some embodiments, determining the compatibility threshold of any posting may include using a classification model. A classification model may be configured to input collected data and cluster data to a centroid based on, but not limited to, frequency of appearance of the range of versioning numbers, linguistic indicators of compatibility and/or allowability, and the like. Centroids may include scores assigned to them such that the compatibility threshold may each be assigned a score. In some embodiments, a classification model may include a K-means clustering model. In some embodiments, a classification model may include a particle swarm optimization model. In some embodiments, determining a compatibility threshold may include using a fuzzy inference engine. A fuzzy inference engine may be configured to map one or more compatibility threshold using fuzzy logic. In some embodiments, a plurality of computing devices may be arranged by a logic comparison program into compatibility arrangements. A “compatibility arrangement” as used in this disclosure is any grouping of objects and/or data based on skill level and/or output score. Membership function coefficients and/or constants as described above may be tuned according to classification and/or clustering algorithms. For instance, and without limitation, a clustering algorithm may determine a Gaussian or other distribution of questions about a centroid corresponding to a given compatibility threshold and/or version authenticator, and an iterative or other method may be used to find a membership function, for any membership function type as described above, that minimizes an average error from the statistically determined distribution, such that, for instance, a triangular or Gaussian membership function about a centroid representing a center of the distribution that most closely matches the distribution. Error functions to be minimized, and/or methods of minimization, may be performed without limitation according to any error function and/or error function minimization process and/or method as described in this disclosure.

Still referring to FIG. 6, inference engine may be implemented according to input of time series inputs 108 and physiological indicator 120. For instance, an acceptance variable may represent a first measurable value pertaining to the classification of time series inputs 108 to physiological indicator 120. Continuing the example, an output variable may represent physiological indicator 120 associated with the user. In an embodiment, time series inputs 108 and/or physiological indicator 120 may be represented by their own fuzzy set. In other embodiments, the classification of the data into physiological indicator 120 may be represented as a function of the intersection two fuzzy sets as shown in FIG. 6, An inference engine may combine rules, such as any semantic versioning, semantic language, version ranges, and the like thereof. The degree to which a given input function membership matches a given rule may be determined by a triangular norm or “T-norm” of the rule or output function with the input function, such as min (a, b), product of a and b, drastic product of a and b, Hamacher product of a and b, or the like, satisfying the rules of commutativity (T(a, b)=T(b, a)), monotonicity: (T(a, b)≤T(c, d) if a≤c and b≤d), (associativity: T(a, T(b, c))=T(T(a, b), c)), and the requirement that the number 1 acts as an identity element. Combinations of rules (“and” or “or” combination of rule membership determinations) may be performed using any T-conorm, as represented by an inverted T symbol or “⊥,” such as max(a, b), probabilistic sum of a and b (a+b−a*b), bounded sum, and/or drastic T-conorm; any T-conorm may be used that satisfies the properties of commutativity: ⊥(a, b)=⊥(b, a), monotonicity: ⊥(a, b)≤⊥(c, d) if a≤c and b≤d, associativity: ⊥(a, ⊥(b, c))=⊥(⊥(a, b), c), and identity element of 0. Alternatively or additionally T-conorm may be approximated by sum, as in a “product-sum” inference engine in which T-norm is product and T-conorm is sum. A final output score or other fuzzy inference output may be determined from an output membership function as described above using any suitable defuzzification process, including without limitation Mean of Max defuzzification, Centroid of Area/Center of Gravity defuzzification, Center Average defuzzification, Bisector of Area defuzzification, or the like. Alternatively or additionally, output rules may be replaced with functions according to the Takagi-Sugeno-King (TSK) fuzzy model.

A first fuzzy set 604 may be represented, without limitation, according to a first membership function 608 representing a probability that an input falling on a first range of values 612 is a member of the first fuzzy set 604, where the first membership function 608 has values on a range of probabilities such as without limitation the interval [0,1], and an area beneath the first membership function 608 may represent a set of values within first fuzzy set 604. Although first range of values 612 is illustrated for clarity in this exemplary depiction as a range on a single number line or axis, first range of values 612 may be defined on two or more dimensions, representing, for instance, a Cartesian product between a plurality of ranges, curves, axes, spaces, dimensions, or the like. First membership function 608 may include any suitable function mapping first range 612 to a probability interval, including without limitation a triangular function defined by two linear elements such as line segments or planes that intersect at or below the top of the probability interval. As a non-limiting example, triangular membership function may be defined as:

$$( x , a , b , c ) = \{ \begin{matrix}0 , & for x > c and x < a \\ \frac{x - a}{b - a} , & for a \leq x < b \\ \frac{c - x}{c - b} , & if b < x \leq c\end{matrix}$$

a trapezoidal membership function may be defined as:

$$y ( x , a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- a ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c , ) = {[ 1 + {\vert \frac{x - c}{a} \vert}^{2 b} ]}^{- 1}$$

Persons skilled in the art, upon reviewing the entirety of this disclosure, will be aware of various alternative or additional membership functions that may be used consistently with this disclosure.

First fuzzy set 604 may represent any value or combination of values as described above, including any time series inputs 108 and physiological indicator 120. A second fuzzy set 616, which may represent any value which may be represented by first fuzzy set 604, may be defined by a second membership function 620 on a second range 624; second range 624 may be identical and/or overlap with first range 612 and/or may be combined with first range via Cartesian product or the like to generate a mapping permitting evaluation overlap of first fuzzy set 604 and second fuzzy set 616. Where first fuzzy set 604 and second fuzzy set 616 have a region 636 that overlaps, first membership function 608 and second membership function 620 may intersect at a point 632 representing a probability, as defined on probability interval, of a match between first fuzzy set 604 and second fuzzy set 616. Alternatively or additionally, a single value of first and/or second fuzzy set may be located at a locus 636 on first range 612 and/or second range 624, where a probability of membership may be taken by evaluation of first membership function 608 and/or second membership function 620 at that range point. A probability at 628 and/or 632 may be compared to a threshold 640 to determine whether a positive match is indicated. Threshold 640 may, in a non-limiting example, represent a degree of match between first fuzzy set 604 and second fuzzy set 616, and/or single values therein with each other or with either set, which is sufficient for purposes of the matching process; for instance, the classification into one or more query categories may indicate a sufficient degree of overlap with fuzzy set representing time series inputs 108 and physiological indicator 120 for combination to occur as described above. Each threshold may be established by one or more user inputs. Alternatively or additionally, each threshold may be tuned by a machine-learning and/or statistical process, for instance and without limitation as described in further detail below.

In an embodiment, a degree of match between fuzzy sets may be used to rank one resource against another. For instance, if both time series inputs 108 and physiological indicator 120 have fuzzy sets, physiological indicator 120 may be generated by having a degree of overlap exceeding a predictive threshold, processor 104 may further rank the two resources by ranking a resource having a higher degree of match more highly than a resource having a lower degree of match. Where multiple fuzzy matches are performed, degrees of match for each respective fuzzy set may be computed and aggregated through, for instance, addition, averaging, or the like, to determine an overall degree of match, which may be used to rank resources; selection between two or more matching resources may be performed by selection of a highest-ranking resource, and/or multiple notifications may be presented to a user in order of ranking.

Referring to FIG. 7, an illustrative embodiment of a systems for reliable uncertainty-aware active regression with imbalanced medical time series. Automated analysis of medical time series, (e.g., ECG), can serve as a critical diagnostic and suggestive tool that allows experts to make fast and efficient decisions. Medical time series have been hypothesized to be related to or predictive of many physiological indicators used in downstream diagnosis. Deep neural networks (DNNs) can be used to process such signals effectively for classification (e.g., providing a binary or categorical output). However, in many real-world scenarios, estimating an actual continuous value of a physiological indicator can be more valuable to the end users (e.g., medical professionals performing the diagnosis). In real-world, the data for such signals can be highly skewed or imbalanced, especially towards normal cases if the prevalence of the abnormal condition or condition of interest is low. For example, a dataset may have a high frequency of healthy or normal measurements and a low frequency of unhealthy or abnormal measurements. A heavily skewed or imbalanced dataset 132 can lead to trained DNNs that are inaccurate in the prediction or estimate of a continuous value and unable to correctly quantify uncertainty of any such predictions at deployment. This can occur if the dataset used to train the DNN lack sufficient samples across the range of continuous values. Thus, it is desirable to develop DNNs that can accommodate such imbalanced datasets 132 and quantify uncertainty of estimated continuous values (point-estimates). Additionally, real-world deployment often involves updating of a trained model as new patient data becomes available to consistently perform well, but labeling the newly available data is costly and time-intensive. Therefore, an efficient method to select a small but highly informative subset of new data is desirable.

Disclosed herein are systems and methods (1) to improve the point estimates of continuous values from time-series data using deep neural networks while simultaneously estimating the uncertainty in the point-estimates or estimates of continuous values; (2) to perform precise calibration of the derived uncertainty estimates; (3) to incorporate the highly calibrated uncertainty estimates in the decision making process leading to significant improvements in quality of the final decisions; and (4) to incorporate the highly calibrated uncertainty estimates to create new patient cohorts (e.g., new training and validation sets) in unknown environments to improve the quality of the model predictions in such situations in an efficient manner.

A dataset for a medical time series can include any dataset where a value is measured over time. One example of a medical time series is an electrocardiogram (ECG), which measures voltage over time to monitor the heart's electrical activity. Nonlimiting examples of medical times series include electrocardiogram (ECG), electroencephalogram (EEG), pulse oximetry, and blood pressure.

A continuous value can include any physiological value or biomarker that can be estimated or predicted from a medical time series. For example, an ECG time series can be used to estimate the level of potassium in the blood, left ventricular ejection fraction, blood pressure, and systolic pressure.

It can be beneficial to use a time series to estimate a continuous value because many types of times series can be collected non-invasively and simply, whereas some continuous values require more time-consuming or invasive tests. For example, an ECG is a time series that can be measured non-invasively and continuously. In this example, ECG can be used to measure levels of electrolytes, e.g., potassium in the blood. Measuring potassium in the blood typically requires drawing a patient's blood, which is more invasive and more difficult to measure continuously. By using a time series to estimate a continuous value, that value can be estimated over time, without having to measure that continuous value repeatedly.

The systems and methods disclosed herein can include one or more of the following steps shown in FIG. 7. Each of these steps is described in further detail below. First, given an imbalanced medical time series training set, a kernel density estimate (e.g., a Gaussian kernel density estimate) is fit (1). This estimate can be used to quantify the imbalance so that it can be accounted for when training a model. For example, this estimate can be used later to reweight the contribution of the samples in the loss function such that less frequent samples contribute more to an estimate. Second, an uncertainty-aware regression model, e.g., a DNN, can be trained to simultaneously produce point-estimates of the continuous value and the associated uncertainty in those estimates (2). Third, a validation set can be used to create hyperfine bins based on predicted point-estimates of the continuous values of physiological indicator and to perform uncertainty scaling to calibrate the uncertainty in each of those fine bins. In some embodiments, a validation set is small and separate from training-set (3). Fourth, a lightweight boosted ensemble of weak learners (e.g., decision trees) can be trained using the predicted point estimates as well as the uncertainty to make a final decision or assign a sample to a standardized range or class (4). Such learners can support clinical decision making. This ensemble can be trained from all the samples in training and validation sets or from any subset thereof. In some embodiments this process does not involves any change to the point estimates or uncertainty estimates. Fifth, in an unknown novel environment with newly available data, the top-K samples can be selected based on the uncertainty values to create a mixture of old training data and the most-uncertain newly labeled data to fine tune the model (5).

In some embodiments, First, a training set 701 of time series data is labeled with measured continuous values (810). A validation set 702 is similarly labeled with measured continuous values (820). The labeled training set 701 is used to train a model 703 (730). The trained mode 703 can then be used to produce estimated continuous values and uncertainty for the validation set 702 (740).

In some embodiments, shown in FIG. 7, the validation set 702 can then be used to calibrate the uncertainty estimation by the trained model 703 (750). The uncertainty estimation can be calibrated by creating hyperfine bins 704 and placing each sample from the validation set 802 into a bin based on the estimated continuous value (751). Then, a bin-wise scaling factor is determined for each bin 704 by optimization (752). This scaling factor can then be used to calibrate the uncertainty for samples within each bin. In some embodiments, this scaling factor can be used to improve estimates of uncertainty by the model 703, for example, by making the uncertainty estimates more reliable at deployment. In some embodiments, uncertainty estimates without a scaling factor (uncalibrated estimates) are less reliable.

In some embodiments, shown in FIG. 7, the model 703 can be updated or fine tuned when an unknown, new, unlabeled data set 705 is added (760). The trained model 703 is run on the new data set 705 to calculate the estimated continuous value and uncertainty for each sample. A subset of K samples with the highest uncertainty is selected to create a new/uncertain dataset 706 (761). The training set 701 and the new/uncertain data set 706 can then be combined to create a mixed dataset 707 (762), and this mixed data set 707 can be used to finetune the model 703 (763).

In some embodiments, a labeled training and a validation set of medical time series (e.g., ECGs) are provided along with a continuous value label for each time series. In some embodiments, a continuous value label can be obtained by additional lab tests. These datasets can be represented by:

$${D}_{train} = {\{ {x}_{i} , {y}_{i} \}}_{i = 1 : N} , {D}_{valid} = {\{ {x}_{i} , {y}_{i} \}}_{i = 1 : M}$$

### Where,

xᵢ represents the medical time series.
yᵢ represents the continuous value groundtruth label.
N represents the total number of samples in the training set.
M represents the total number of samples in the validation set.

In some embodiments, the dataset can be imbalanced. In an imbalanced dataset 132, certain kinds of time series and continuous values, {xᵢ, yᵢ}, may not be well represented in the dataset. For example, an imbalanced dataset 132 can be identified by checking a distribution of the ground truth target values to see if any range of values are over-represented or under-represented. Disclosed herein are methods to simultaneously estimate the continuous value yᵢ (represented by ŷᵢ) and the uncertainty estimate (represented by \hat{σ}ᵢ) from a time series (e.g., as shown in step 2 of FIG. 1). Also disclosed herein are uncertainty calibration techniques that can provide hyperfine calibrated uncertainty estimates (represented by \hat{σ}_{calib,i}) that work by solving an optimization problem in hyperfine bins created on the basis of predicted continuous values for the samples in a validation set (D_{valid}). In some embodiments, the validation set is a labeled data set used to identify the stop point of training and to calibrate the uncertainty from a trained model. In some embodiments, the validation set is small.

Also disclosed herein are methods to use the continuous value estimate and the calibrated uncertainty-estimate to improve the decision making or to fine-tune the model when new data sets are added. For example, given an unseen, new, unlabeled dataset consisting of the medical time series,

$${D}_{n o v e l} = {\{ {x}_{i} \}}_{i = 1 : P}$$

Where P may be large and thus labeling all the samples is infeasible, the systems and methods disclosed herein can leverage the trained model's predictions to create a smaller subset of K samples to further refine the model in a way that leads to larger gains in model performance than randomly choosing the samples from the new dataset.

The systems and methods disclosed herein include optimization for improved continuous value point and uncertainty estimation. In some embodiments, a model or estimation function ψ(·; θ) for the continuous value is learned using deep neural network parameterized by parameters θ. In these embodiments, the model is trained using a training set that is labeled, e.g., with a measured continuous value for each time series sample. In some embodiments, a training set is selected randomly from the dataset. Before training the model, the training set can be fit (e.g., with a Gaussian kernel density estimate) so that samples can be weighted appropriately to account for an imbalanced dataset 132 during optimization and training. In these embodiments, the function Y can take a medical time series as the input and predict a continuous value and corresponding uncertainty estimate. That is,

$$Ψ ( x ; θ ) = \{ \hat{y} , \hat{σ} \}$$

In some embodiments, the model is trained by solving one of the optimization problems below. For example, the optimal parameters of the deep neural network ψ, represented by θ*, can be obtained by solving the following optimization problem

$${θ}^{*} = arg {min}_{θ} \frac{1}{N} {\sum }_{i = 1}^{N} ( {λ}_{1} * ( \frac{1}{{ρ ( {y}_{i} )}^{{λ}_{2}}} \vert {[ Ψ ( {x}_{i} ; θ ) ]}_{\hat{y}} - {y}_{i} \vert ) + {λ}_{3} * ( \frac{{\vert {[ Ψ ( {x}_{i} ; θ ) ]}_{\hat{y}} - {y}_{i} \vert}^{2}}{{[ Ψ ( {x}_{i} ; θ ) ]}_{\hat{σ}}^{2}} + log ( {[ Ψ ( {x}_{i} ; θ ) ]}_{\hat{σ}}^{2} ) ) )$$

### Where,

ρ(y) is the Gaussian kernel density estimate (a fit on the training data labels, e.g., as shown in step 1 of FIG. 1) for the target value y.
[ψ(xᵢ; θ)_{ŷ}], is the ŷᵢ estimate given by the network.
[ψ(xᵢ; θ)]_{\hat{σ}} is the σ̅ᵢ estimate given by the network.
λ₁, λ₂, λ₃ are the hyperparameters.

In the above optimization, the term

$$( \frac{1}{{ρ ( {y}_{i} )}^{{λ}_{2}}} \vert {[ Ψ ( {x}_{i} ; θ ) ]}_{\hat{y}} - {y}_{i} \vert )$$

computes the discrepancy between the continuous value prediction and the groundtruth (labeled continuous value), where the discrepancy is weighted lower if the groundtruth label (labeled continuous value) is present in abundance, e.g., density ρ(y) is high, and the discrepancy is weighted higher if the label density is low, addressing the imbalanced nature of the dataset. In this way, the weights can account for imbalance in the training set.

In the above optimization, the term

$$( \frac{{\vert {[ Ψ ( {x}_{i} ; θ ) ]}_{\hat{y}} - {y}_{i} \vert}^{2}}{{[ Ψ ( {x}_{i} ; θ ) ]}_{\hat{σ}}^{2}} + log ( {[ Ψ ( {x}_{i} ; θ ) ]}_{\hat{σ}}^{2} ) )$$

indicates negative-log-likelihood of heteroscedastic Gaussian distribution assumed for each prediction.

In the above optimization, the hyperparameters λ₁, λ₃ control the relative contribution of a density-based discrepancy term and the heteroscedastic negative-log-likelihood term that learns to estimate the uncertainty. In the above optimization, λ₂ is a hyper parameter that helps in scaling the weight for the loss term of each sample.

In another example, a more generalized form of the parametric distribution to estimate the heteroscedastic uncertainty can be used. For example, instead of modeling the residuals as Gaussian distribution, one can model the residuals as Generalized Gaussian distribution (GGD). A GGD can better model a heavy tailed distribution. In this example, optimization problem then becomes

$${θ}^{*} = arg {min}_{θ} \frac{1}{N} {\sum }_{i = 1}^{N} ( {λ}_{1} * ( \frac{1}{{ρ ( {y}_{i} )}^{{λ}_{2}}} \vert {[ Ψ ( {x}_{i} ; θ ) ]}_{\hat{y}} - {y}_{i} \vert ) + {λ}_{3} * ( \frac{{\vert {[ Ψ ( {x}_{i} ; θ ) ]}_{\hat{y}} - {y}_{i} \vert}^{{[ Ψ ( {x}_{i} . θ ) ]}_{\hat{β}}}}{{[ Ψ ( {x}_{i} ; θ ) ]}_{\hat{α}}^{{[ Ψ ( {x}_{i} ; θ ) ]}_{\hat{β}}}} - log ( \frac{{[ Ψ ( {x}_{i} ; θ ) ]}_{\hat{β}}}{{[ Ψ ( {x}_{i} ; θ ) ]}_{\hat{α}}} ) + log ( Γ ( \frac{1}{{[ Ψ ( {x}_{i} ; θ ) ]}_{\hat{β}}} ) ) ) )$$

### Where,

ρ(y) is the Gaussian kernel density estimate (fit on the training labels) for the target value y.
[ψ₍ₓ_{ᵢ}_{; θ)}]_{ŷ} is the ŷᵢ estimate given by the network.
[ψ₍ₓ_{ᵢ}_{; θ)}]_{\hat{α}} is the \hat{α}ᵢ estimate given by the network, representing the scale parameter of GGD
[ψ₍ₓ_{ᵢ}_{; θ)}]_{\hat{β}} is the \hat{β}ᵢ estimate given by the network, representing the shape parameter of GGD
λ₁, λ₂, λ₃ are the hyperparameters.
The uncertainty estimate will be given by,

$$\frac{{[ {Ψ}_{( {x}_{i} ; 0 )} ]}_{\hat{α}} \sqrt{3 {/ [ Ψ ( {x}_{i} ; θ ) ]}_{\hat{β}}}}{\sqrt{1 / [ {Ψ}_{{( {x}_{i} ; θ )}_{\hat{β}}}}}$$

In some embodiments, the above optimization problems can be solved using a deep neural network and back-propagation algorithm.

The systems and methods disclosed herein can also include post-training hyperfine uncertainty calibration. In some embodiments, once the deep neural network ψ is trained, e.g., after the optimal parameters θ* have been found as described in the above optimization step, the deep neural network ψ(·; θ*) can estimate both the target continuous value and the uncertainty in the prediction. However, in some instances, the uncertainty estimates obtained can be biased estimates and not well calibrated. For example, uncertainty estimates can be biased because they are based on estimates of the continuous value, rather than the ground truth continuous value. Additionally, because the optimization occurs on a training set, the uncertainty estimates can overfit to the training set, rather than being generalized to other datasets.

Therefore, disclosed herein are systems and methods to calibrate the uncertainty estimates. In some embodiments, global optimal scaling factor s* are first found by solving the following optimization problem on the validation set D_{valid}, where the parameters of the neural network are fixed to the optimal obtained above (θ*)

$${s}^{*} = arg {min}_{s} ( M log ( s ) + \frac{1}{2 {s}^{2}} {\sum }_{i = 1}^{M} ( \frac{{\vert {[ Ψ ( {x}_{i} ; {θ}^{*} ) ]}_{\hat{y}} - {y}_{i} \vert}^{2}}{{[ Ψ ( {x}_{i} ; {θ}^{*} ) ]}_{\hat{σ}}^{2}} ) )$$

In some embodiments, this optimization minimizes the difference between the estimated uncertainty (\hat{σ}ₜ) and the actual difference between the estimated continuous value and the ground truth continuous value (ŷᵢ-yᵢ).

The model is then run in inference mode to generate the estimates for all the samples in the validation set, providing a new augmented validation set,

$${D}_{valid - aug} = {\{ {x}_{i} , {y}_{i} , {\hat{y}}_{i} , {\hat{σ}}_{i} \}}_{i = 1 : M}$$

Hyperfine bins of uniform length δ are then created spanning the range of predicted continuous values, e.g.,

$$b = int ( \frac{{\hat{y}}_{max} - {\hat{y}}_{min}}{δ} )$$

bins are created, where δ<<ŷₘₐₓ-ŷₘᵢₙ leading to hyperfine bins, where ŷₘₐₓ=max({ŷᵢ}_{i=1:M}) and ŷₘᵢₙ=min({ŷᵢ}_{i=1:M}). δ can be selected so that it is much smaller than ŷₘₐₓ-ŷₘᵢₙ. In some embodiments, δ can be selected so that b is at least 100 or at least 1000. The n^{th} bin being Bₙ spanning [ŷₘᵢₙ+(n−1)*δ, ŷₘᵢₙ+n*δ]. In this example, each sample in the validation set then assigned to one of the bins based on its estimated continuous value.

Then, a bin-wise scaling factor is found for each of the above hyperfine bins by solving an optimization problem. Let the bin-wise scaling factor for bin Bₙ be ηₙ given by,

$${{η}_{n} = arg {min}_{η} ( len ( \{ \vert {\hat{y}}_{i} - {y}_{i} \vert \leq η * {s}^{*} * {\hat{σ}}_{i} \}}_{{\hat{y}}_{i} ∈ {B}_{n}} ) / len ( {B}_{n} ) \geq ζ ) ∀ {\hat{y}}_{i} ∈ {B}_{n}$$

### Where,

len(·) is the function that gives the number of elements in the input set
ζ represents the minimum acceptable fraction in each bin where the discrepancy between the prediction and the groundtruth (label) is captured by the scaled uncertainty estimates
   In some embodiments, this optimization minimizes the difference between the estimated uncertainty (\hat{σ}ᵢ) and the actual difference between the estimated continuous value and the ground truth continuous value (ŷᵢ-yᵢ)

In some embodiments, the above scaling global and bin-wise factors allow computation of well calibrated uncertainty estimates for each predicted continuous value. For example, given input x, the continuous value prediction (ŷ) and the calibrated uncertainty (\hat{σ}_{calib}) are given by,

$$\begin{matrix}\{ \hat{y} , \hat{σ} \} = Ψ ( x ; {θ}^{*} ) \\ {\hat{σ}}_{calib} = {η}_{B i n ( \hat{y} )} * {s}^{*} * \hat{σ}\end{matrix}$$

### Where,

Bin(ŷ) is the hyperfine bin to which ŷ belongs
η_{Bin(ŷ)} is the bin-wise scaling factor for the hyperfine bin Bin(ŷ)

The systems and methods disclosed herein can also include improved decision making using continuous value point and uncertainty estimates, e.g., with gradient boosted corrections. These methods are shown, for example, in step 4 of FIG. 1. In some embodiments, decisions based on a predicted continuous value that help the medical professional in diagnosing the condition are based on the predicted value lying in a certain standardized range. For example, a doctor can base a diagnosis on whether a continuous value falls within a given standardized range. In one example, potassium level in blood is represented by a number between 0 and 14. In this example there are standardized ranges, including 0-3.5 (very low, medical attention required), 3.5-5.5 (normal), and >5.5 (very high, medical attention needed. Disclosed herein are systems and methods that utilize both the predicted continuous value and calibrated uncertainty estimate to infer a standardized range. In some embodiments, such an estimate is better than relying on just the continuous value prediction. For example, such an estimate accounts for uncertainty and can allow a human to disregard a highly uncertain prediction from the model that is likely to be wrong

In some embodiments, these systems and methods use predictions (both ŷ, \hat{σ}_{calib}) from the trained deep neural network (ψ) as input features to an ensemble of decision trees that uses gradient-boosting technique to learn a random forest (represenated by Y) that infers the standardized-range in which the predicted continuous value belongs, accounting for the uncertainty in the predictions, for example

$$γ ( x ) = γ ( {[ Ψ ( x ; {θ}^{*} ) ]}_{\hat{y}} , {η}_{B i n ( {[ Ψ ( x ; {θ}^{*} ) ]}_{\hat{y}} )} * {s}^{*} * {[ Ψ ( x ; {θ}^{*} ) ]}_{\hat{σ}} ) = Standardized ‐ range label$$

Although a single decision tree can be used, a gradient-boosted ensemble can provide better results. In some embodiments, the decision trees are readily available for use. In some embodiments the decision trees are not specific to a particular continuous value or type of time series

In some embodiments, the random forest is trained on dataset Dₜᵣₐᵢₙ∪D_{valid}. (the union of the training and validation sets). In some embodiments, the random forest is trained on the training set, the validation set, or any combination thereof.

The systems and methods disclosed herein can also include leveraging hyperfine calibrated uncertainty for active learning. In some embodiments, systems and methods disclosed herein leverage the hyperfine calibrated uncertainty to improve a deep neural network model in a novel environment, e.g., when new, unlabeled data is added. In some embodiments, a deep neural network ψ is trained with an initial labeled dataset Dₜᵣₐᵢₙ (validation set being D_{valid}), leading to ψ(·; θ*), which is a first version of the model. In some circumstances, at a later point in time, a huge amount of unlabeled data can become available, for example, from an unseen new environment (e.g., data from different hardware, different healthcare facilities, different demographics). In these circumstances, it is desirable to re-train or fine-tune the first version of the model on this new dataset as well to improve the generalizability of the model. The unseen, new, unlabeled dataset can be represented by Dₙₒᵥₑₗ={xᵢ}_{i=1:P} where P is large enough such that labeling all the P samples may be infeasible or undesirable due to resource constraints. However, some labeling is needed to retrain or fine-tune the first version of the model. In some embodiments, active learning can be used to choose a small subset of K(<<P) samples to label that will yield a larger gain in performance when used for retraining or fine-tuning a previous model compared to using a random subset of K samples. In some embodiments, K can be selected based on the resources or budgets available for labeling the dataset. In some embodiments, active learning can provide a larger gain in performance compared to random selection (e.g., if the underlying data is imbalanced). For example, active learning can be used to select a subset of samples more likely to lead to a gain in model performance.

In some embodiments, the subset of K samples is selected based on calibrated uncertainty estimates. In these embodiments, the first version of the model ψ(·; θ*) is run in inference mode on all the P samples from Dₙₒᵥₑₗ to create a set of tuples D_{novel-unc}, where a tuple includes the input samples and corresponding calibrated uncertainty estimated using the first version of the model as described above:

$${D}_{n o v e l - u n c} = {\{ {x}_{i} , {\hat{σ}}_{c a l i b , i} \}}_{i = 1 :} p$$

In these embodiments, the samples can be arranged in a list of sorted samples based on decreasing calibrated uncertainty value to identify the top-K samples with the most uncertainty. In some embodiments, a subset of the new data set having the K most uncertain samples are selected for labelling:

$${D}_{l a b e l - n o v e l} = {\{ {x}_{i} , {y}_{i} \}}_{i = 1 : K} ∀ {x}_{i} ∈ topK ( {D}_{n o v e l - u n c} )$$

Where, topK(D_{novel-unc}) represents the K most uncertain samples from Dₙₒᵥₑₗ.

In some embodiments, the model (e.g., the deep neural network) is then re-trained or fine-tuned on dataset Dₘᵢₓ=Dₜᵣₐᵢₙ∪D_{label}-novel yielding a next improved version of the model. In some embodiments, the model retrained or fine tuned on the subset of the new dataset or a combination of the subset of the new dataset and the training set.

### Examples

With continued reference to FIG. 7, certain embodiments will now be described in the following non-limiting examples in the following paragraphs. A dataset was curated at a medical site and included over 2.5 million patients, with over 8.5 million 12-lead ECGs coming from different types of hardware, covering a wide range of medical conditions, age, and demographic variables. The data collection is from year 1980 to 2020.

The proposed framework was applied to three tasks (i) Estimating age from a given ECG, (ii) Estimating survival from a given ECG, (iii) Estimating Blood Potassium level from a given ECG, (iv) Estimating Left Ventricular Ejection Fraction (LVEF) from a given ECG. FIG. 3 shows the results for these three tasks. The graph for individual task shows that the predicted distribution from the framework largely encompasses the distribution representing the groundtruth values, indicating that the predicted point and uncertainty estimates are accurate and well-calibrated. For a given point on x-axis: (i) Red bold curve shows the peak of the groundtruth distribution (the distribution of groundtruth values that correspond to predicted value given by the x-axis). (ii) Shaded red region indicates the spread of the groundtruth distribution (i.e., 2 and 98 percentile of the groundtruth distribution). (iii) Blue bold curve shows the peak of the predicted distribution. (iv) Blue shaded region indicates the spread of the groundtruth distribution (i.e., 2 and 98 percentile of the groundtruth distribution). The predicted distribution from the model tightly encompasses groundtruth distribution indicating predictions are well-calibrated. The model uncertainty was calibrated using small validation sets but calibration generalizes well to much larger test sets.

Estimating Age from ECG: Certain patterns/features (e.g., amplitude and duration of certain waves, as well as changes in the intervals between waves) of the ECG waveform can change with age, and the ECG waveform can be used to estimate an individual's cardiovascular age. For these experiments, a large and diverse dataset of ECGs (along with the patient's age) was curated. 6.7/0.75/1.35 million train/val/test datasets were used.

Estimating Survival (time to death) from ECG: ECG has potential for non-invasive survival prediction to aid in the diagnosis and treatment of life-threatening conditions. For these experiments, the data was curated to include only patients for which the death has been recorded. The time interval from ECG recording to death for such patients provide groundtruth for survival (time-to-death). In total there were 3 million ECGs with the corresponding time-to-death, and 2.3/0.26/0.44 million train/val/test sets were used.

Estimating Level of Potassium in Blood from ECG: Serum potassium level is a measure of the amount of potassium in the blood. Potassium is an electrolyte that is important for the proper functioning of the body's cells, tissues, and organs. It helps regulate the balance of fluids in the body, maintain normal blood pressure, and support proper muscle function, among other functions. The normal range for serum potassium levels is typically considered to be between 3.5-5.0 millimoles per liter (mmol/L). Levels below 3.5 mmol/L are considered to be low (hypokalemia) and levels above 5.0 mmol/L are considered to be high (hyperkalemia). Both low and high potassium levels can have serious health consequences. While a blood test is used to measure the serum potassium level, machine learning techniques can be used to infer the potassium level using ECGs, for example, for remote patient monitoring. In total there are over 4 million ECGs with the corresponding serum potassium level (derived using blood test), 2.1/0.4/1.7 million train/val/test sets were used.

Estimating Left Ventricular Ejection Fraction from ECG: Low left ventricular ejection fraction (LVEF) is a condition in which the left ventricle of the heart is unable to pump a sufficient amount of blood out of the heart and into the body. The left ventricle is the main pumping chamber of the heart and is responsible for pumping oxygenated blood to the rest of the body. LVEF is typically measured as a percentage, with a normal range being between 55-70%. A LVEF below 40% is generally considered to be low and may indicate heart failure. LVEF can be evaluated through various non-invasive tests, including an echocardiogram or a nuclear stress test. But ECG can still provide valuable information about heart function and can be used in conjunction with other tests to assess LVEF and diagnose heart conditions. In these experiments, DNNs were leveraged infer the LVEF. This can help reduce the costs associated with overall diagnosis and treatment by reducing the need for more expensive Echocardiogram. In total there are over 0.6 million ECGs with the corresponding LVEF (derived using Echo), and 0.3/0.1/0.2 million train/val/test sets were used.

To perform a comparative study with 12-lead ECG time series, a backbone DNN architecture was created that adapts 2D ResNets into a ID version (with 1D convoluted layers). The DNN backbone architecture was used to create (i) class of deterministic regression models, (ii) class of uncertainty aware probabilistic regression models, and (iii) class of classification models for some of the tasks. For the probabilistic regression the backbone architecture is appended with two smaller feed-forward heads to estimate the parameters of the predictive distribution. The deterministic regression model is trained with (i) squared error (L2) loss, termed as Regress.-L2, (ii) absolute error (L1) loss, termed as Regress.-L1, (iii) The density based weights were incorporated to modulate the loss terms, called Regress.-L1-KDEw. Two variants of probabilistic regression models were trained (i) a standard heteroscedastic Gaussian model that estimates the mean and variance for the prediction and trained to maximize the likelihood, called (Regres.-w.-U) (an uncertainty-aware, but uncalibrated model), and (ii) the proposed model (HypUC) that incorporates the calibration technique along with gradient-boosted corrections (an uncertainty aware, calibrated model). For serum potassium level estimation (and LVEF estimation), the model was also compared with a binary classifier that predicts whether the given ECG has Hyperkalemia (and low LVEF for LVEF estimation).

Table 1 shows the performance of various methods on different tasks and datasets. For all regression tasks, Mean Squared Error (MSE) and Mean Absolute Error (MAE) were computed to measure the discrepancy between the predictions and the groundtruth. Moreover, Spearman and Pearson correlation coefficient indicate the positive association between the predicted point estimates from the network and the groundtruth target variable. High correlation indicates that the prediction will be able to indicate the severity of the medical condition (which maybe helpful in prescribing the treatment as explained in Section 1). For probabilistic models, the quality of uncertainty estimates is measured by Uncertainty Calibration Error (UCE) and Negative Log-Likelihood (NLL). Finally, for the binary classification tasks, the following parameters were also reported: Area under the Curve (AUC), Sensitivity, Specificity, Positive Predictive Values (PPV), and Negative Predictive Values (NPV).

TABLE 1
Quantitative results on 4 different tasks (Survival, Age, Serum Potassium, and
Left Ventricular Ejection Fraction Estimation) showing the performance of various
deterministic and probabilistic regression and classification models.

| | | | | Spear. | Pears. | | | | | | | |
|---|---|---|---|---|---|---|---|---|---|---|---|---|
| Tasks | Methods | MSE | MAE | Corr. | Corr. | UCE | NLL | AUC | Sensiti. | Specifi. | PPV | NPV |
| Survival | Regres. -L2 | 100.12 | 8.49 | 0.3 | 0.3 | NA | NA | NA | NA | NA | NA | NA |
| Estimation | Regres. -L1 | 73.21 | 7.78 | 0.38 | 0.41 | NA | NA | NA | NA | NA | NA | NA |
| | Regres -L1- | 67.97 | 6.3 | 0.51 | 0.53 | NA | NA | NA | NA | NA | NA | NA |
| | KDEw | | | | | | | | | | | |
| | Regres -w. -U | 84.62 | 8.13 | 0.35 | 0.38 | 2.37 | 5.89 | NA | NA | NA | NA | NA |
| | HypUC | 54.62 | 5.38 | 0.56 | 0.61 | 0.58 | 3.45 | NA | NA | NA | NA | NA |
| Age | Regres. -L2 | 151.69 | 9.47 | 0.58 | 0.6 | NA | NA | NA | NA | NA | NA | NA |
| Estimation | Regres. -L1 | 116.52 | 7.93 | 0.73 | 0.75 | NA | NA | NA | NA | NA | NA | NA |
| | Regres. -L1- | 99.77 | 7.64 | 0.78 | 0.81 | NA | NA | NA | NA | NA | NA | NA |
| | KDEw | | | | | | | | | | | |
| | Regres -W. - U | 136.27 | 8.22 | 0.68 | 0.72 | 13.32 | 7.84 | NA | NA | NA | NA | NA |
| | HypUC | 74.9 | 6.7 | 0.84 | 0.88 | 1.06 | 3.7 | NA | NA | NA | NA | NA |
| Serum | Binary- | NA | NA | 0.39 | 0.41 | NA | NA | 0.87 | 0.74 | 0.85 | 0.1 | 0.99 |
| Potassium | Classifier | | | | | | | | | | | |
| Estimation | Regres -L2 | 0.26 | 0.52 | 0.51 | 0.54 | NA | NA | 0.84 | 0.7 | 0.86 | 0.1 | 0.99 |
| | Regres. -L1 | 0.19 | 0.34 | 0.52 | 0.54 | NA | NA | 0.86 | 0.7 | 0.87 | 0.09 | 0.99 |
| | Regres. -L1- | 0.19 | 0.33 | 0.52 | 0.55 | NA | NA | 0.86 | 0.72 | 0.85 | 0.11 | 0.99 |
| | KDEw | | | | | | | | | | | |
| | Regres -w.-U | 0.23 | 0.38 | 0.51 | 0.55 | 1.83 | 8.7 | 0.86 | 0.71 | 0.86 | 0.1 | 0.99 |
| | HypUC | 0.2 | 0.32 | 0.52 | 0.55 | 0.41 | 3.12 | 0.89 | 0.77 | 0.86 | 0.1 | 0.99 |
| Ejection | Binary- | NA | NA | 0.28 | 0.32 | NA | NA | 0.91 | 0.77 | 0.87 | 0.09 | 0.98 |
| Fraction | Classifier | | | | | | | | | | | |
| Estimation | Regres. -L2 | 171.37 | 13.56 | 0.37 | 0.39 | NA | NA | 0.9 | 0.75 | 0.84 | 0.09 | 0.98 |
| | Regres. -L1 | 153.66 | 11.78 | 0.41 | 0.42 | NA | NA | 0.91 | 0.76 | 0.86 | 0.1 | 0.98 |
| | Regres. -L1- | 141.62 | 10.71 | 0.41 | 0.43 | NA | NA | 0.91 | 0.75 | 0.87 | 0.09 | 0.99 |
| | KDEw | | | | | | | | | | | |
| | Regres. -w.-U | 165.81 | 12.24 | 0.41 | 0.44 | 10.56 | 9.83 | 0.9 | 0.74 | 0.85 | 0.1 | 0.99 |
| | HypUC | 133.26 | 10.21 | 0.43 | 0.45 | 1.68 | 4.28 | 0.93 | 0.77 | 0.87 | 0.1 | 0.99 |

As indicated in Table 1, for Survival Estimation, the regression model trained with L2 loss (Regres.-L2) has a MSE of 100.12 (MAE of 8.49, Spearman correlation of 0.30 and Pearson correlation of 0.30), which are worse than the performance of the model trained with L1 loss (Regres.-L1) (MSE of 73.21, MAE of 7.78, Spearman correlation of 0.38 and Pearson correlation of 0.41). Moreover, the performance of the regression model trained with L1 loss could be further improved by incorporating kernel density-based weights (Regres.-L1-KDEw) that account for imbalance in the dataset and leads to a performance with MSE of 67.97 (MAE of 6.30, Spearman correlation of 0.51, and Pearson correlation of 0.53). Additionally, uncertainty-aware, uncalibrated heteroscedastic Gaussian probabilistic regression model (Regres.-w.-U) performs better than homoscedastic Gaussian model (Regress.-L2) but not as good as (Regress.-L1). Regres.-w.-U estimates the uncertainty in the prediction (with UCE/NLL of 2.37/5.89), but the proposed uncertainty-aware, calibrated HypUC model not only improves regression performance (with MSE/MAE of 54.62/5.38) but the quality of uncertainty as well (with UCE/NLL of 0.58/3.45). A similar trend was observed for the Age Estimation, where the proposed uncertainty-aware, calibrated HypUC model performs the best.

For Serum Potassium Estimation, the proposed uncertainty-aware, calibrated HypUC model was compared with a binary classifier baseline, an approach found in previous work, which predicts if a given ECG corresponds to Hyperkalemia or not, instead of estimating the serum potassium level. While such a binary classifier can achieve a good classification performance as indicated by an AUC of 0.87, it does not gauge the severity of the condition that may decide the diagnosis. A correlation between the ground truth serum potassium level compared to the scores may be derived from binary classifier (left and middle), the lack of high positive correlation (Spearman correlation of 0.30) indicates that the scores from the model cannot be used as an indication for the severity of the medical condition (within Hyperkalemia) but can be used to discriminate Hyperkalemia from non-Hyperkalemia cases. Different kinds of regression models were also trained to directly regress the serum potassium level. As indicated in Table 1, regression models generally produce outputs that are better correlated with the groundtruth (e.g., Regress.L2 has a Spearman correlation coefficient of 0.51 vs. 0.39 for Binary Classifier). Moreover, among deterministic regression models Regres.-L1-KDEw performs the best with MSE of 0.19 (MAE of 0.33). The uncertainty-aware but uncalibrated probabilistic regressor Regres.-w.-U performs better than Regres.-L2 and provides uncertainty estimates as well (UCE/NLL of 1.83/8.76). But, the proposed uncertainty-aware, calibrated HypUC model provides the best regression performance, along with better uncertainty estimates. Additionally, the outputs from the regression models can be used to classify the ECG into Hyperkalemia or not (similar to binary classifier). All the regression models perform comparable classification to classifier. In particular, the HypUC model has AUC of 0.89 which is higher than the binary classifier (AUC of 0.87).

A similar trend is observed for Left Ventricular Ejection Fraction (LVEF) Estimation where the proposed uncertainty aware, calibrated HypUC model performs well in regression along with providing better calibrated uncertainty estimates. Moreover, when used as a classifier, HypUC also yields a better performance than the binary classifier trained from scratch, emphasizing the benefits of tackling this problem with the proposed probabilistic regression framework, HypUC.

An entropy-based technique to tag unreliable predictions was applied to estimations of survival, age, serum potassium, and LVEF. A threshold τ_{q,val} that was computed using the validation set as the q-% tile value from the distribution of entropy values for LVEF. A threshold of the 10% percentile such that predictions with entropy above the 10% percentile are considered unreliable. FIG. 6 evaluates the regression performance of proposed model HypUC (in terms of MAE) on the test-sets for various tasks (survival estimation, age estimation, serum potassium estimation, left ventricular ejection estimation) after removing the unreliable predictions based on different thresholds derived from the validation set. As a higher fraction of unreliable samples is removed, (decreasing the threshold τ_{q,val} by lowering the value of q) the performance improves. This demonstrates that the predictions made by model can be enhanced by only showing reliable predictions and flagging the unreliable predictions (based on entropy measure). Flagged predictions can be evaluated by human experts who may be critical for deploying machine learning models for healthcare applications in real-world.

It will be appreciated that while one or more particular materials or steps have been shown and described for purposes of explanation, the materials or steps may be varied in certain respects, or materials or steps may be combined, while still obtaining the desired outcome. Additionally, modifications to the disclosed embodiment and the invention as claimed are possible and within the scope of this disclosed invention.

Those of skill in the art would appreciate that the various illustrations in the specification and drawings described herein can be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware, software, or a combination depends upon the particular application and design constraints imposed on the overall system. Skilled artisans can implement the described functionality in varying ways for each particular application. Various components and blocks can be arranged differently (for example, arranged in a different order, or partitioned in a different way) all without departing from the scope of the subject technology.

Furthermore, an implementation of the communication protocol can be realized in a centralized fashion in one computer system, or in a distributed fashion where different elements are spread across several interconnected computer systems. Any kind of computer system, or other apparatus adapted for carrying out the methods described herein, is suited to perform the functions described herein.

A typical combination of hardware and software could be a general purpose computer system with a computer program that, when being loaded and executed, controls the computer system such that it carries out the methods described herein. The methods for the communications protocol can also be embedded in a non-transitory computer-readable medium or computer program product, which includes all the features enabling the implementation of the methods described herein, and which, when loaded in a computer system is able to carry out these methods. Input to any part of the disclosed systems and methods is not limited to a text input interface. For example, they can work with any form of user input including text and speech.

Computer program or application in the present context means any expression, in any language, code or notation, of a set of instructions intended to cause a system having an information processing capability to perform a particular function either directly or after either or both of the following a) conversion to another language, code or notation; b) reproduction in a different material form. Significantly, this communications protocol can be embodied in other specific forms without departing from the spirit or essential attributes thereof, and accordingly, reference should be had to the following claims, rather than to the foregoing specification, as indicating the scope of the invention.

The communications protocol has been described in detail with specific reference to these illustrated embodiments. It will be apparent, however, that various modifications and changes can be made within the spirit and scope of the disclosure as described in the foregoing specification, and such modifications and changes are to be considered equivalents and part of this disclosure.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, systems, methods and media for carrying out the several purposes of the disclosed subject matter. It is important, therefore, that the claims be regarded as including such equivalent constructions insofar as they do not depart from the spirit and scope of the disclosed subject matter.

It will be appreciated that while one or more particular materials or steps have been shown and described for purposes of explanation, the materials or steps may be varied in certain respects, or materials or steps may be combined, while still obtaining the desired outcome. Additionally, modifications to the disclosed embodiment and the invention as claimed are possible and within the scope of this disclosed invention.

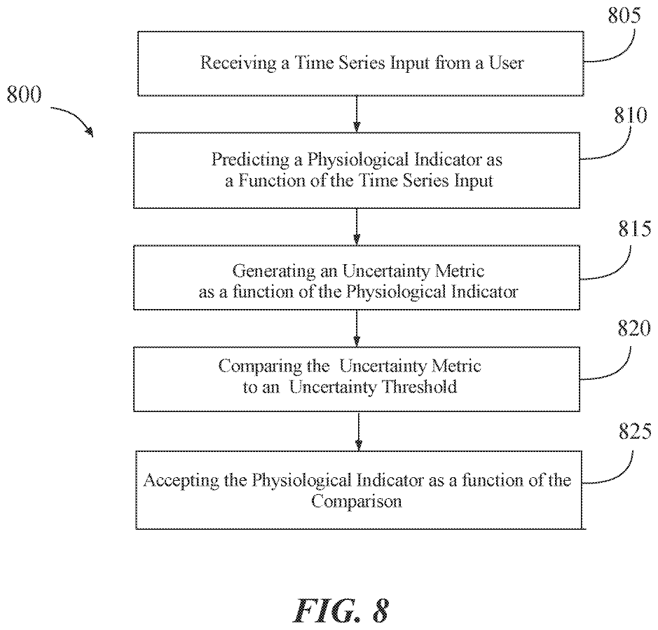

Referring now to FIG. 8, a flow diagram of an exemplary method 800 for predicting a physiological indicator is illustrated. At step 805, method 800 includes receiving, using at least a processor, a time series input from a user. This may be implemented as described and with reference to FIGS. 1-7. In an embodiment, the method may include receiving a plurality electrocardiogram signals from a plurality of sensors.

Still referring to FIG. 8, at step 810, method 800 includes predicting, using the at least a processor, a physiological indicator as a function of the time series input. This may be implemented as described and with reference to FIGS. 1-7. Predicting the physiological indicator includes receiving a plurality of prediction training data, wherein the plurality of prediction training data includes a plurality of time series inputs correlated to examples of physiological indicators as outputs, wherein the examples of physiological indicators include continuous value labels. Predicting the physiological indicator includes fitting the plurality of prediction training data with a kernel density estimate. Predicting the physiological indicator includes training the prediction machine learning model using a plurality of prediction training data, wherein training the prediction machine learning model comprises using the kernel density estimate to weight a contribution of each time series input of the plurality of prediction training data based on an abundance of the corresponding continuous value label. Predicting the physiological indicator includes determining the physiological indicator as a function of the time series input using a trained prediction machine learning model. In an additional embodiment, the physiological indicator may include a measurement of the left ventricular ejection fraction (LVEF) or level of potassium in the blood of the user.

Still referring to FIG. 8, at step 815, method 800 may include generating, using the at least a processor, an uncertainty metric as a function of the physiological indicator. This may be implemented as described and with reference to FIGS. 1-7.

Still referring to FIG. 8, at step 820, method 800 may include comparing, using the at least a processor, the uncertainty metric to an uncertainty threshold. This may be implemented as described and with reference to FIGS. 1-7.

Still referring to FIG. 8, at step 825, method 800 may include accepting, using the at least a processor, the physiological indicator as a function of the comparison. This may be implemented as described and with reference to FIGS. 1-7. In an embodiment, the method may include generating, using the at least a processor, diagnostic data as a function of the acceptance of the physiological parameter.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

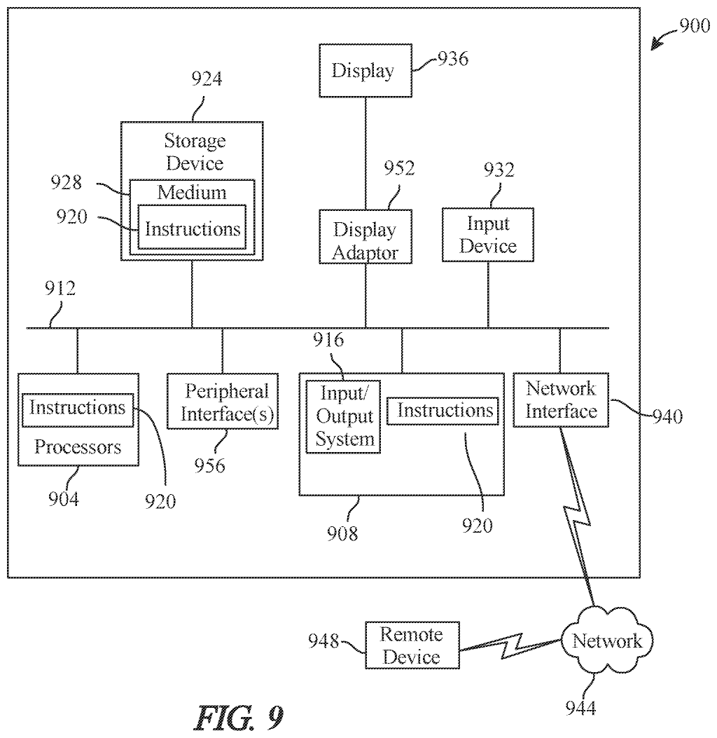

FIG. 9 shows a diagrammatic representation of one embodiment of a computing device in the exemplary form of a computer system 900 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 900 includes a processor 904 and a memory 908 that communicate with each other, and with other components, via a bus 912. Bus 912 may include any of several types of bus structures including, but not limited to, a memory bus, a memory controller, a peripheral bus, a local bus, and any combinations thereof, using any of a variety of bus architectures.

Processor 904 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 904 may be organized according to Von Neumann and/or Harvard architecture as a non-limiting example. Processor 904 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 908 may include various components (e.g., machine-readable media) including, but not limited to, a random-access memory component, a read only component, and any combinations thereof. In one example, a basic input/output system 916 (BIOS), including basic routines that help to transfer information between elements within computer system 900, such as during start-up, may be stored in memory 908. Memory 908 may also include (e.g., stored on one or more machine-readable media) instructions (e.g., software) 920 embodying any one or more of the aspects and/or methodologies of the present disclosure. In another example, memory 908 may further include any number of program modules including, but not limited to, an operating system, one or more application programs, other program modules, program data, and any combinations thereof.

Computer system 900 may also include a storage device 924. Examples of a storage device (e.g., storage device 924) include, but are not limited to, a hard disk drive, a magnetic disk drive, an optical disc drive in combination with an optical medium, a solid-state memory device, and any combinations thereof. Storage device 924 may be connected to bus 912 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 924 (or one or more components thereof) may be removably interfaced with computer system 900 (e.g., via an external port connector (not shown)). Particularly, storage device 924 and an associated machine-readable medium 928 may provide nonvolatile and/or volatile storage of machine-readable instructions, data structures, program modules, and/or other data for computer system 900. In one example, software 920 may reside, completely or partially, within machine-readable medium 928. In another example, software 920 may reside, completely or partially, within processor 904.

Computer system 900 may also include an input device 932. In one example, a user of computer system 900 may enter commands and/or other information into computer system 900 via input device 932. Examples of an input device 932 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 932 may be interfaced to bus 912 via any of a variety of interfaces (not shown) including, but not limited to, a serial interface, a parallel interface, a game port, a USB interface, a FIREWIRE interface, a direct interface to bus 912, and any combinations thereof. Input device 932 may include a touch screen interface that may be a part of or separate from display 936, discussed further below. Input device 932 may be utilized as a user selection device for selecting one or more graphical representations in a graphical interface as described above.

A user may also input commands and/or other information to computer system 900 via storage device 924 (e.g., a removable disk drive, a flash drive, etc.) and/or network interface device 940. A network interface device, such as network interface device 940, may be utilized for connecting computer system 900 to one or more of a variety of networks, such as network 944, and one or more remote devices 948 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 944, may employ a wired and/or a wireless mode of communication. In general, any network topology may be used. Information (e.g., data, software 920, etc.) may be communicated to and/or from computer system 900 via network interface device 940.

Computer system 900 may further include a video display adapter 952 for communicating a displayable image to a display device, such as display device 936. Examples of a display device include, but are not limited to, a liquid crystal display (LCD), a cathode ray tube (CRT), a plasma display, a light emitting diode (LED) display, and any combinations thereof. Display adapter 952 and display device 936 may be utilized in combination with processor 904 to provide graphical representations of aspects of the present disclosure. In addition to a display device, computer system 900 may include one or more other peripheral output devices including, but not limited to, an audio speaker, a printer, and any combinations thereof. Such peripheral output devices may be connected to bus 912 via a peripheral interface 956. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. An apparatus for predicting a physiological indicator, wherein the apparatus comprises:
at least a processor; and
a memory communicatively connected to the at least a processor, wherein the memory containing instructions configuring the at least a processor to:
receive a time series input;
predict a physiological indicator as a function of the time series input, wherein predicting the physiological indicator comprises:
receiving a plurality of prediction training data, wherein the plurality of prediction training data comprises a plurality of time series inputs correlated to examples of physiological indicators as outputs, wherein the examples of physiological indicators comprise continuous value labels;
fitting the plurality of prediction training data with a kernel density estimate;
training the prediction machine learning model using a plurality of prediction training data, wherein training the prediction machine learning model comprises using the kernel density estimate to weight a contribution of each time series input of the plurality of prediction training data based on an abundance of the corresponding continuous value label; and
determining the physiological indicator as a function of the time series input using a trained prediction machine learning model.

2. The apparatus of claim 1, wherein receiving the time series input comprises receiving a plurality electrocardiogram signals from a plurality of sensors.

3. The apparatus of claim 1, wherein the memory contains instructions further configuring the at least a processor to retrain the prediction machine learning model, wherein retraining the prediction machine-learning model comprises:
receive a set of novel time-series inputs;
determine an uncertainty metric for each value of the set of novel time-series inputs using the prediction machine-learning model; and
choose a subset of the novel time-series inputs, wherein choosing the subset comprises choosing values of the set of novel-time series inputs as a function of the uncertainty metric.

4. The apparatus of claim 3, wherein retraining the prediction machine-learning model further comprises:
labeling the subset of novel time-series inputs; and
retraining the prediction machine-learning model using the labeled subset of novel time-series inputs.

5. The apparatus of claim 4, wherein labeling the subset of novel time-series data comprises labeling the subset of novel time-series inputs using active learning.

6. The apparatus of claim 1, wherein the physiological indicator comprises a measurement of the left ventricular ejection fraction (LVEF).

7. The apparatus of claim 1, wherein the physiological indicator comprises a measurement of the level of potassium in the blood of the user.

8. The apparatus of claim 1, wherein the memory contains instructions further configuring the at least a processor to calibrate an uncertainty metric for the prediction machine-learning model, wherein calibrating the uncertainty metric of the prediction machine-learning model comprises:
receiving a set of validation data;
sorting each datapoint of the validation set into a plurality of hyperfine bins as a function of a physiological indicator; and
determining a bin-wise scaling factor for each of the plurality of hyperfine bins.

9. The apparatus of claim 1, wherein the memory further instructs the processor to generate diagnostic data as a function of the acceptance of the physiological parameter.

10. The apparatus of claim 1, wherein fitting the plurality of prediction training data with the kernel density estimate further comprises selecting a bandwidth of the kernel density estimate.

11. A method for predicting a physiological indicator, wherein the method comprises:
receiving, using at least a processor, a time series input;
predicting, using the at least a processor, a physiological indicator as a function of the time series input, wherein predicting the physiological indicator comprises:
receiving a plurality of prediction training data, wherein the plurality of prediction training data comprises a plurality of time series inputs correlated to examples of physiological indicators as outputs, wherein the examples of physiological indicators comprise continuous value labels;
fitting the plurality of prediction training data with a kernel density estimate;
training the prediction machine learning model using a plurality of prediction training data, wherein training the prediction machine learning model comprises using the kernel density estimate to weight a contribution of each time series input of the plurality of prediction training data based on an abundance of the corresponding continuous value label; and
determining the physiological indicator as a function of the time series input using a trained prediction machine learning model.

12. The method of claim 11, wherein receiving the time series input comprises receiving a plurality electrocardiogram signals from a plurality of sensors.

13. The method of claim 11, wherein the method further comprises retraining the prediction machine learning model, wherein retraining the prediction machine-learning model comprises:
receiving, using at least a processor, a set of novel time-series inputs;
determining, using at least a processor, an uncertainty metric for each value of the set of novel time-series inputs using the prediction machine-learning model; and
choosing, using at least a processor, a subset of the novel time-series inputs, wherein choosing the subset comprises choosing values of the set of novel-time series inputs as a function of the uncertainty metric.

14. The method of claim 13, wherein retraining the prediction machine-learning model further comprises:
labeling, using at least a processor, the subset of novel time-series inputs; and
retraining, using at least a processor, the prediction machine-learning model using the labeled subset of novel time-series inputs.

15. The method of claim 14, wherein labeling the subset of novel time-series data comprises labeling the subset of novel time-series inputs using active learning.

16. The method of claim 11, wherein the physiological indicator comprises a measurement of the left ventricular ejection fraction (LVEF).

17. The method of claim 11, wherein the physiological indicator comprises a measurement of the level of potassium in the blood of the user.

18. The method of claim 11, wherein the method further comprises calibrating, using at least a processor, an uncertainty metric for the prediction machine-learning model, wherein calibrating the uncertainty metric of the prediction machine-learning model comprises:
receiving a set of validation data;
sorting each datapoint of the validation set into a plurality of hyperfine bins as a function of a physiological indicator; and
determining a bin-wise scaling factor for each of the plurality of hyperfine bins.

19. The method of claim 11, wherein the method further comprises generating, using at least a processor, diagnostic data as a function of the acceptance of the physiological parameter.

20. The method of claim 11, wherein fitting the plurality of prediction training data with the kernel density estimate further comprises selecting a bandwidth of the kernel density estimate.
